(19) United States
(12) Patent Application Publication
(10) Pub. No.: US 2023/0366023 A1
(43) Pub. Date: Nov. 16, 2023

(54) METHODS AND COMPOSITIONS FOR BIOLUMINESCENCE-BASED SEQUENCING

(71) Applicant: BGI Shenzhen Co., Ltd., Shenzhen, Guangdong (CN)

(72) Inventors: Yan Chen, Sunnyvale, CA (US); Handong Li, San Jose, CA (US); Yongwei Zhang, Saratoga, CA (US)

(21) Appl. No.: 18/246,959

(22) PCT Filed: Oct. 9, 2021
(86) PCT No.: PCT/CN2021/122738

§ 371 Date: Mar. 28, 2023

Related U.S. Application Data
(60) Provisional application No. 63/089,996, filed on Oct. 9, 2020.

(51) Int. Cl. C12Q 1/6874

(52) U.S. Cl. CPC C12Q 1/6874

(57) ABSTRACT

Disclosed herein are methods and compositions for corresponding positions on an array with differently labeled affinity reagents immobilized at the positions. A first and a second affinity reagents are immobilized on at least some of the first and second positions, respectively. The first affinity reagent and the second affinity reagent are associated with a first luciferase polypeptide and a second luciferase polypeptide, respectively. The first luciferase polypeptide does not cross react with the second substrate and the second luciferase polypeptide does not cross react with the first substrate. The method further comprises contacting the array with the first substrate, which reacts with the first luciferase polypeptide and detecting the first luminescent signal and detecting the second luminescent signal at positions, thereby corresponding positions on an array with the first affinity reagent or the second affinity reagent immobilized at the positions.

### RELATED APPLICATION

This application claims priority to U.S. Provisional Application No. 63/089,996, filed Oct. 9, 2020, the entire content of which is incorporated by reference.

### SEQUENCE LISTING

The instant application contains a Sequence Listing which has been submitted electronically in ASCII format and is hereby incorporated by reference in its entirety. Said ASCII copy, created on Sep. 23, 2021, is named 092171-1260984_(5092-WOCN)_SL.txt and is 3,535 bytes in size.

### FIELD

This application relates to methods and compositions for bioluminescence-based nucleic acid sequencing.

### BACKGROUND

US. Pat. Pub. No. 20180223358 describes a massively parallel sequencing (MPS) chemistry (CooIMPS™) that employs non-labeled reversible terminator (NLRT) nucleotides and antibodies that specifically recognize and distinguish NLRTs having different nucleobases. Compared to the conventional MPS chemistry, CooIMPS™ sequencing uses a non-labeled reversible terminator instead of dye-labeled reversible terminator, which allows further extension of the strand in a new cycle of sequencing without any interference from the prior cycle. Furthermore, NLRTs are easier and less costly to make, and they can be incorporated more efficiently. Another advantage of CooIMPS™ is that antibodies can allow more flexible modification and carry multiple signaling molecules to improve sequencing signal compared with single dye per base on standard labeled RTs.

Luciferases and their substrates have been used in reporter technologies. Luciferases react with (e. g., oxidize) their substrates and emits light signal, which can be captured and quantified. These light-emitting reactions are widely used in in vitro and in vivo food testing, environmental monitoring and diagnostics. Coelenterazine is one of the common substrates that can be oxidized by a large number of different luciferases. Aubin Fleiss & Karen S. Sarkisyan, A brief review of bioluminescent systems (2019), Current Genetics volume 65, pages 877-882(2019), available at: link.springer.com/article/10.1007/s00294-019-00951-5.

### SUMMARY OF INVENTION

In one aspect, disclosed herein is a method of corresponding positions on an array with differently labeled affinity reagents immobilized at the positions. The method comprises providing an array of positions wherein the array comprises first positions and second positions, wherein a first affinity reagent is immobilized at least some of the first positions and a second affinity reagent is immobilized at least some of the second positions. The first affinity reagent is associated with a first luciferase polypeptide and the second affinity reagent is associated with a second luciferase polypeptide. The first luciferase polypeptide can react with a first substrate to generate a first luminescent signal, and the second luciferase polypeptide can react with a second substrate to generate a second luminescent signal. The first substrate is orthogonal to the first luciferase polypeptide and the second substrate is orthogonal to the second luciferase polypeptide. The first luciferase polypeptide does not have significant cross-substrate reactivity with the second substrate and the second luciferase polypeptide does not have significant cross-substrate reactivity with the first substrate. The method further comprises contacting the array with the first substrate and detecting the first luminescent signal at positions at which the first affinity reagent is immobilized; contacting the array with the second substrate and detecting the second luminescent signal at positions at which the second affinity reagent is immobilized. The method further comprises determining at a position the first affinity reagent is immobilized if the first luminescent signal is detected at said position or determining at a position the second affinity reagent is immobilized if the second luminescent signal is detected at said position.

In another aspect, disclosed herein is a kit for performing sequencing, the kit comprising a first protein, a second protein, a first substrate, and a second substrate. The first protein is associated with a first luciferase polypeptide, and the first luciferase polypeptide can specifically react with a first substrate to generate a first luminescent signal. The second protein is associated with a second luciferase polypeptide, and the second luciferase polypeptide can specifically react with a second substrate to generate a second luminescent signal. The first luciferase polypeptide does not have significant cross-substrate reactivity with the second substrate (which is simply referred to as the first luciferase polypeptide does not cross react with the second substrate throughout this disclosure), and the second luciferase polypeptide does not have significant cross-substrate reactivity with the first substrate (which is simply referred to as the second luciferase does not cross react with the first substrate throughout this disclosure).

In yet another aspect, disclosed herein is a method of producing a luciferase-antibody conjugate, comprising: (1) providing (i) an antibody that specifically recognizes a 3′-O-reversible terminator deoxyribonucleotides comprising a nucleobase that is selected from the group consisting of adenine (A), cytosine (C), guanine (G), thymine (T), and analogs thereof; and (ii) a luciferase polypeptide; (2) contacting the luciferase polypeptide with 2-iminothiolane under conditions that generate an —SH group on the luciferase polypeptide, thereby producing a luciferase polypeptide comprising an —SH group; (3) contacting the antibody with an SMCC, wherein the —NHS group of the SMCC is linked to the —NH2 group on the antibody, thereby producing an SMCC-linked antibody having a maleimide group, and (4) contacting the luciferase polypeptide comprising the —SH group with the SMCC-linked antibody under conditions suitable for protein conjugation thereby forming the luciferase-antibody conjugate.

### BRIEF DESCRIPTION OF THE DRAWINGS

FIG. 1 shows various luciferases and their orthogonal substrates that can be used in the methods and compositions disclosed herein.

FIG. 2 compares signals from various commercial luciferases.

FIG. 3 shows there is no significant cross-substrate reactivity between Gluc and Nluc.

FIG. 4 shows f-CTZ is the optimal substrate for the Nluc luciferase used herein.

FIG. 5 shows the onboard stability (room temperature stability for 24 hours) of CTZ and f-CTZ is not significantly different from the stability under the fresh condition (newly prepared).

FIG. 6A and FIG. 6B show the long-term (e.g., up to 6 months) stability of the orthogonal pair of Gluc/CTZ and Nluc/f-CTZ.

FIG. 7A illustrates the linking chemistry between Nluc and SMCC. FIG. 7B illustrates the chemistry of an antibody reacts with Tris (2-carboxyethyl) phosphine hydrochloride (TCEP) to generate sulfhydryl groups (—SH), which can be used to link SMCC.

FIG. 8 shows gel electrophoresis results, which show the formation of antibody-Nluc conjugates and antibody-Gluc conjugates. Lane 1: molecular latter; Lane 2: a A-Nluc (clone 1867, multiple bands shown between 160-260 KDa); Lane 3: a T-Nluc; lane 4: a G-Gluc; lane 5: a DIG-Nluc.

FIG. 9A-9C show the signals produced by Nluc-anti DIG conjugates, Nluc-anti T antibody conjugates, and Nluc-anti A antibody conjugates detected on an MGI DNBseq E series one-color sequencer imager.

FIG. 10A illustrates the formation of a covalent linkage between the luciferase polypeptide and the SMCC linker. FIG. 10B shows the results of the signals produced by Gluc and Nluc after the free SH group on the luciferases are blocked by Iodoacetamide.

FIG. 11A shows the reaction of treating Gluc and Nluc with the Traut's reagent. FIG. 11B shows the reaction for conjugating the antibody to an SMCC linker. FIGS. 11C and 11D show the signal intensity and thermostability of Gluc and Nluc that have been modified with Traut's reagent before being conjugated to the antibodies.

FIG. 12A shows the protein yield after incubating antibody with reducing agent TCEP at the various ratios, after 100 KDa cutoff purification. FIG. 12B shows the —SH group labeling degree by the Ellman quantification method. FIG. 12C shows the images acquired by the sequencer imager after treating the antibody with various reducing agents at different ratios.

FIG. 13A-13D show the imaging results of using biotinylated secondary antibody specifically for the isotype NLRT antibodies and strepavidin labeled Gluc. FIG. 13A-13D detect the presence of C-biotin, anti-A antibody, anti-T antibody, and anti-G antibody, respectively.

FIG. 14 shows the the binding capacity of the antibody-luciferase fusion proteins to the 3′-O-reversible terminator deoxyribonucleotide.

FIG. 15 shows imaging signal produced by NLRT antibodies, each has been been conjugated to biotin through an EZ-NHS-S-S-Biotin linker.

FIG. 16 shows an exemplary cool MPS™ bioluminescence one color sequencing scheme.

FIG. 17A shows signal histogram from first five cycle of sequencing of the methods as described in FIG. 16. FIG. 17B shows the images 1 and 2 from the first cycle of the sequencing. FIG. 17C-17E show the signal, noise, and SNR plot for each cycle.

FIG. 18A shows that no significant changes in the signal from Nluc upon DTT treatment. FIG. 18B shows that the signal from Gluc reduced more than 95% upon DTT treatment.

FIG. 19A-19E show the signals from the first five sequencing cycles. The results show good signal separation from background. In each signal scattering plot, the X axis indicates the normalized signal intensity from Image 1, and the Y axis indicates the normalized signal intensity from Image 2. There were four (4) well separated signal groups obtained from this scattering plots. The one on the X axis (median=0.5, 0) is from the biotin-labeled nucleotide A. The one on the Y axis (0, median=0.5) is from the Digoxin-labeled nucleotide C. Signal from the (0, 0) position indicates signal from nucleotide G, which was unlabeled. Signal from the (0.5, 0.5) position in the 45 degree is signal from the nucleotide T since it was half labeled with Biotin and half with Digoxin. So one half of the Ts was lighted up in image 1 and the other half lighted up in Image 2. In each cycle of the signal scattering plot, these four (4) signal groups were well differentiated from each other, rendering it feasible for correct base call for that cycle.

### DETAILED DESCRIPTION OF THE INVENTION

### Glossary

The term “CTZ” refers to coeleterazine.

The term “f-CTZ” refers to fluorinated coeleterazine.

As used herein, the term “substantially the same” when referring to the activity of two luciferases means that when reacting with the same substrate, the difference in signals generated by the two luciferases is less than 40%, less than 30%, less than 20%, less than 10% of the values of the lesser signal.

The term “Gluc” refers to the Gaussia luciferase polypeptide (SEQ ID NO: 1) or a variant thereof, provided the variant is substantially identical in amino acid sequence to Gluc (SEQ ID NO: 1) and also has substantially the same activity as Gluc (SEQ ID NO: 1).

The term “Nluc” refers to NanoZac luciferase polypeptide (SEQ ID NO: 2) or a variant thereof, provided the variant is substantially identical in amino acid sequence to Nluc (SEQ ID NO: 2) and also has substantially the same activity as Nluc (SEQ ID NO: 2).

The term “NLRT” refers to a 3′-O-reversible terminator deoxyribonucleotide.

The term “Traut's reagent” refers to a type of cyclic thioimidate compound for thiolation (sulfhydryl addition), known as 2-iminothiolane or 2-IT. Traut's Reagent reacts with primary amines (—NH2) to introduce sulfhydryl (—SH) groups while maintaining charge properties similar to the original amino group.

The term “on-board stability” refers to the stability of luciferase polypeptides in reaction buffer under the room temperature for 24 hours.

The term “orthogonal substrate” refers to the substrate that can be cleaved by a specified luciferase polypeptide. For example, f-CTZ is the orthogonal substrate for the Nluc mutant described herein below, and CTZ is the orthogonal substrate for the Gluc mutant described herein below. A luciferase and its orthogonal substrate together are referred to as an orthogonal pair, or an orthogonal enzyme/substrate pair.

The term “reacts with,” refers to that a luciferase contacts a substrate and converts (e.g., oxidizes) the substrate into a new molecule in a light-emitting chemical reaction.

The term “GDS,” refers to a primer extension strand, e.g., the DNA strand that is formed by incorporating NLRTs during the sequencing reaction. GDS is also known as “primer extension product,” or “extended primer.”

The term “corresponding,” as used in “a method of corresponding positions on an array with labeled affinity reagents,” refers to determing a relationship (or correspondence) between a specified physical position on the array and a characteristic (e.g., structure) of an affinity reagent immobilized on or located on the specified array position. Thus, in an array comprising a plurality of physical positions and a plurality of different affinity reagents each immobilized at a position, the identity of the affinity reagent immobilized on any specified position can be determined.

The term “differently labeled,” when referring to two affinity reagents, means that these two affinity reagents are capable of producing distinguishable signals. For example, an antibody labeled with Gluc and an antibody labeled with Nluc are two differently labeled affinity reagents, because the signal produced by Gluc (reacting with its orthogonal substrate CTZ) and the signal produced by Nluc (reacting with its orthogonal subtrate f-CTZ) are distinguishable.

### Overview

The present invention use luciferases conjugated to affinity reagents, such as antibodies, that bind to each NLRT having a distinct nucleobase, and methods of using the conjugates in massively parallel bioluminescence-based one-color nucleic acid sequencing. Detection of bases in each sequencing cycle using a bioluminescent signal advantageously eliminates the need for excitation source and color filters in the sequencing hardware design.

In one approach the sequencing methods use at least two differently labeled affinity reagents that can specifically bind to two different 3′-O-reversible terminator deoxyribonucleotides. The two affinity reagents are associated with (e.g., conjugated to or bound to) two different luciferase polypeptides, and the two different luciferase polypeptides can react with their respective orthogonal substrates to produce high luminescent signals. For illustration and not limitation, one exemplary luciferase polypeptide is NanoZac luciferase polypeptide (Nluc) (Prolume Inc.). An orthogonal substrate for Nluc, as discussed below, is fluroinated coeleterazine (f-CTZ). Another exemplary luciferase polypeptide is Gaussia luciferase (Gluc) ((Prolume Inc., J. Welsh, Biochemical and Biophysical Research Communications 389 (2009) 563-568, M43L, M110L mutant). An orthogonal substrate for Gluc is coeleterazine (CTZ).

The two luciferase polypeptides used in the method do not have significant cross-substrate reactivity (i.e. have minimal cross talk), which minimize the chance of misreads. In some embodiments, the first luciferase polypeptide is Gluc and the second luciferase polypeptide is Nluc. To further reduce the sequencing error caused by the cross-substrate reactivity between Gluc and Nluc, after the signal from the first luciferase Gluc is detected and recorded, a deactivation agent is applied to the sequencing array to deactivate Gluc. Then the signal from the second luciferase, Nluc, reacting with its orthogonal substrate, is detected and recorded. The deactive agent's activity is selective, i.e., it deactivates Gluc but is not able to deactivate Nluc, i.e., it does not affect the enzymatic activity of Nluc.

Using two luciferase-substrate orthogonal pairs allows a unique, two-label (i.e., two luciferases), one-color sequencing and improves the efficiency and accuracy of one-color sequencing. The methods described herein can be used for a two-label, one-color sequencing scheme, in which two luciferase polypeptides are coupled to two affinity reagents, each binding to a NLRT having a different nucleobase on a sequencing array. The two luciferase polypeptides generate intense luminescent signals when reacting with their respective orthogonal substrates, and they do not have significant cross-substrate reactivity. In one approach two different luciferase-coupled affinity reagents are added to a sequencing array at the same time. The luciferase-coupled affinity reagents bind to a specific polynucleotide incorporated nucleotide and is immobilized at a position (address) in the sequencing array. A first substrate (orthogonal to the first luciferase) may be added and a first signal (produced by the first luciferase cleaving the first substrate) may be detected at specific positions on the array and the information recorded. Then a second substrate may be added to the sequencing array and the second signal (produced by the second luciferase cleaving the second substrate) may be detected/recorded. Nucleotides incorporated at each position of the array can be determined based on the presence or absence of the first or second luminescent signal at that position. Thus, the present method allows for simultaneous delivery of at least two affinity reagents recognizing NLRTs having two different nucleobases during sequencing, and the results can be determined using a sequencer with a single color, or a single channel, imaging system. It significantly reduces the amount of time and labor involved and simplifies the sequencing process. The use of two luciferase-associated affinity reagents may be combined with other labeling/detection methods including the use of four luciferase-associated affinity reagents, as discussed below, the use of nucleotides directly linked (e.g., via a linker) to a fluorescent or bioluminescent signaling system, or any other system.

In one approach methods and compositions disclosed herein may involve four different affinity reagents, each specifically recognizing and binding a NLRT having a different nucleobase. In some approaches, a first affinity reagent is conjugated to a first luciferase polypeptide, a second affinity reagent that is conjugated to a second luciferase polypeptide, a third affinity reagent is conjugated to or bound to the first luciferase and the second luciferase polypeptide, and a fourth affinity reagent is conjugated to or is bound to neither the first luciferase nor the second luciferase polypeptide. Thus, by detecting the first and/or second luminescent signal at a position on the array, one can determine which affinity reagent is bound to the position of the array and thus which NLRT is present on that position.

In some embodiments, the affinity reagent is conjugated to a luciferase polypeptide via a linker. In some embodiments, the luciferase polypeptide is first treated with Traut's reagent (2-iminothiolane) before being coupled to the affinity reagent. It was found surprisingly that the Traut's reagents can increase the enzymatic activity of the luciferase polypeptide and thus increase the signal.

### NLRT

Non-labeled reversible terminators (“NLRT”) are 3′-O-reversible terminator deoxyribonucleotides. 3′-O-reversible terminator deoxyribonucleotides are nucleotide analogs comprising a removable blocking group at the 3′-OH position of the deoxyribose. Although fluorescently labeled reversible terminators are widely used in current sequencing-by-synthesis (SBS) systems, the NLRT used in the current invention may be unlabeled and used in conjunction with anti-nucleotide affinity reagents described herein below. In one embodiment, non-labeled means the NLRT does not comprise a fluorescent dye. In one embodiment, non-labeled means the NLRT does not comprise a chemiluminescent dye. In one embodiment, non-labeled means the NLRT does not comprise a light emitting moiety. Exemplary NLRTs are described in US-2018-0223358-A1, the entire content of which is herein incorporated by reference.

### Array

In some embodiments, the NLRTs are incorporated to the growing DNA strands (“GDS”) that are located on different positions of an array. “Array” means a solid support (or collection of solid supports such as beads) arrayed on a surface, which may be a substantially planar surface, that carries a collection of sites (e.g., an array of wells or derivitized areas on a surface) comprising nucleic acids such that each site of the collection is spatially defined and not overlapping with other sites of the array. The sites can be described as “spatially discrete.” The array can also comprise a non-planar interrogatable structure with a surface such as a bead or a well. The oligonucleotides or polynucleotides of the array may be covalently bound to the solid support, or it may be non-covalently bound. The solid support may be e.g., a bead, flow cell, pad, channel in a microfluidic device and the like) and the solid support may comprise silicon, glass, gold, a polymer, PDMS, and the like. In some embodiments, the template nucleic acid is immobilized or contained within a droplet (optionally immobilized on a bead or other substrate within the droplet).

An array used in the invention comprises template nucleic acids immobilized thereon. In some embodiments, an array includes ordered arrays (meaning template binding regions are arranged in an ordered, typically rectilinear, pattern, such as a grid, spiral, or other patterns), i.e., the identity of templates at any specific position (or “address”) on an array may be known prior to sequencing of the templates. In some embodiments, the array includes disordered arrays (also referred to as random arrays), in which the template binding regions are at random positions, i.e., the identity of a templates at a given address is not known prior to sequencing unknown prior to sequencing reaction.

In some embodiments, the template nucleic acid is an immobilized DNA concatemer comprising multiple copies of a target sequence. In some embodiments, the template nucleic acid is represented as a DNA concatemer, such as a DNA nanoball (DNB) comprising multiple copies of a target sequence and an “adaptor sequence.” See PCT Pat. Pub. WO 2007/133831, the content of which is hereby incorporated by reference in its entirety for all purposes. In some embodiments the template is a single polynucleotide molecule. In some embodiments the template is present as a clonal population of template molecules (e.g., a clonal population produced by bridge amplification or Wildfire amplification).

It will be understood that the method is not limited to a particular form of template, and the template can be any template such as, for example, a DNA concatemer, a dendrimer, a clonal population of templates (e.g., as produced by bridge amplification or Wildfire amplification) or a single polynucleotide molecule. Thus, the specification should be read as if each reference to a template can alternatively refer to a concatemer template, a dendrimer, a clonal population of, e.g., short linear templates, a single molecule template (e.g., in a zero-mode waveguide), and templates in other forms. In some embodiments, the template nucleic acids are DNBs.

In one aspect the invention provides a DNA array comprising: a plurality of template DNA molecules, each DNA molecule attached at a position of the array, a complementary DNA sequence base-paired with a portion of the template DNA molecule at a plurality of the positions, wherein the complementary DNA sequence comprises at its 3′ end an incorporated first reversible terminator deoxyribonucleotide; and a first affinity reagent bound specifically to at least some of the first reversible terminator deoxyribonucleotides, and a second affinity reagent bound specifically to at least some of the second reversible terminator deoxyribonucleotides. The first affinity reagent is conjugated to or bound to a first luciferase polypeptide and the second affinity reagent is conjugated to or bound to a second luciferase polypeptide. The first luciferase polypeptide can specifically react with a first substrate to generate a first luminescent signal, and the second luciferase polypeptide can specifically react with a second substrate to generate a second luminescent signal that is distinguishable from the first luminescent signal. The first luciferase polypeptide does not cross react with the second substrate and the second luciferase polypeptide does not cross react with the first substrate.

### Luciferase Polypeptides and Substrates

A luciferase polypeptide disclosed herein is able to react with its orthogonal substrate to produce high signal intensity and are sufficiently stable. Preferably the luciferase is relatively small (e.g., less than 30 KDa) so that they can be readily engineered, e.g., be coupled to or fused to an affinity reagent disclosed herein. Non-limiting examples of suitable luciferase polypeptides include Gaussia luciferase (GLuc, SEQ ID NO: 1), and NanoZac luciferase (Nluc, SEQ ID NO: 2). Other non-limiting examples of luciferases that can be used include Nanoluc (Promega) and NanoKaz (NanoLight Technology), which also comprise a sequence of SEQ ID NO: 2. As shown in Table 1, as compared to firefly luciferase and Renilla luciferase, Gaussia luciferase (GLuc), Nanoluc luciferase, and NanoKaz luciferase have much higher signal intensity and stability in cells. Thus these luciferases are suitable for the sequencing methods disclosed herein.

TABLE 1
Examples of luciferase polypeptides

| | | Protein | | Signal | Stability | |
|---|---|---|---|---|---|---|
| Luciferase | Substrate | size | | intensity | in cells | |
| firefly | D-Luciferin | 61 | KDa | 1 | 10 | min |
| | (with ADP) | | | | | |
| Renilla | Coelenterazine | 36 | KDa | 1 | 1.7 | hour |
| Gaussia | Coelenterazine | 20 | KDa | 160 | 10.5 | days |
| Nanoluc | Furimazine | 19.1 | KDa | 80 | 7.7 | days |
| NanoKaz | Coelenterazine-F | 19.1 | KDa | 150 | 7.7 | days |

The Gaussia luciferase (GLuc) from the copepod Gaussia princeps is about 20 kDa. GLuc catalyzes the oxidation of coelenterazine to produce an intense blue light at 470 nm. In some embodiments, the Gluc used in this disclosure has a sequence of

(SEQ ID NO: 1)
MKPTENNEDFNIVAVASNFATTDLDADRGKLPGKKLPLEVLKELEANAR
KAGCTRGCLICLSHIKCTPKMKKFIPGRCHTYEGDKESAQGGIGEAIVD
IPEIPGFKDLEPLEQFIAQVDLCVDCTTGCLKGLANVQCSDLLKKWLPQ
RCATFASKIQGQVDKIKGAGGDHHHHHH

It contains two mutations, M43L and M110L (underlined), relative to the wild type Gluc. J. Welsh, Biochemical and Biophysical Research Communications 389 (2009) 563-568, the entire disclosure of which is herein incorporated by reference. As compared to the wild type Gluc, the Gluc (SEQ ID NO: 1) has longer shelf life and retains substantially the same signal intensity as the wild type Gluc.

Nluc is another luciferase that is small (about 19.1 kDa) and highly stable. Nluc is also capable of producing intense luminescence when reacting with its orthogonal substrate. Nluc is derived from the deep-sea shrimp Oplophorus gracilirostris by performing mutagenesis to optimize the luminescence output of the wild type enzyme in the species. See, England et al., Bioconjug. Chem. 2016 may 18 27 (5): 1175-1187, the entire disclosure is herein incorporated by reference. The engineered Nluc is able to produce a signal that is 150 fold higher than the Renilla luciferase (Rluc). In some embodiments, the Nluc used in the method has a sequence of

MVFTLEDFVGDWRQTAGYNLDQVLEQGGVSSLFQNLGVSVTPIQRIVLS
GENGLKIDIHVIIPYEGLSGDQMGQIEKIFKVVYPVDDHHFKVILHYGT
LVIDGVTPNMIDYFGRPYEGIAVFDGKKITVTGTLWNGNKIIDERLINP
DGSLLFRVTINGVTGWRLCERILA (SEQ ID NO: 2), as
described in U.S. Pat. Nos. 9,315,783 and
US9,404,145.

### No Significant Cross-Substrate Reactivity

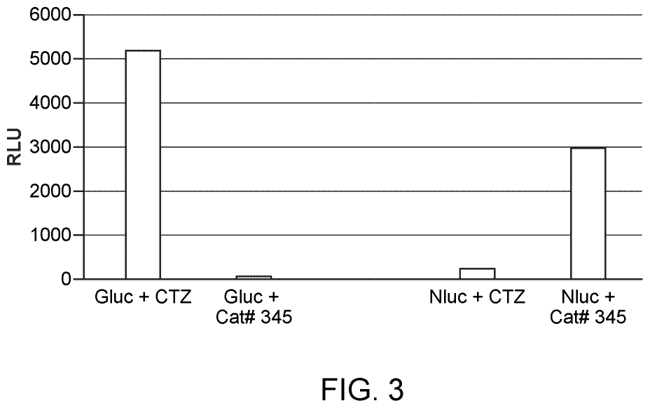

As described below, at least two luciferases are used in various approaches of the one color sequencing scheme disclosed herein. These luciferases do not have significant cross-substrate reactivity thus can minimize noise from sequencing reads. The term “do not have significant cross substrate reactivity,” “or “having minimal crosstalk” refers to the signal intensity produced by a luciferase polypeptide (e.g., the first luciferase polypeptide) reacting with its orthogonal substrate (e.g., the first substrate) is at least 5, at least 6, at least 7, at least 10, at least 50, as least 100, or at least 500 fold greater than the signal intensity produced by the first luciferase polypeptide reacting with a different substrate used in the same reaction (e.g., the second substrate) under the same condition. Accordingly, non-limiting examples of orthogonal pairs that can be used in the invention includes Gaussia luciferase polypeptide (Gluc)/Coeleterazine (CTZ) and NanoZac luciferase polypeptide (Nluc)/Fluorinated coeleterazine (f-CTZ). As shown in FIG. 3 below, Gluc showed only about 2% background signal when reacting with the Nluc substrate, f-CTZ (Cat #345, Nanolight Technology). While Nluc showed only about 10% background signal when reacting with the Gluc substrate, CTZ. This demonstrates that these two enzymes do not have significant cross-substrate reactivity.

Signal produced from a luciferase reacting with its substrate can be detected using any method or device that is capable of detecting luminescence, for example, a luminometer. In some embodiments, the luciferase is incubated with the substrate for a sufficient amount of time to allow the development of the signal before detection. The step of signal development may last between 5 to 30 minutes, for example, about 10 minutes.

It will be understood that other Gluc variants may also be used in the invention, provided that they have substantially the same activity as Gluc (SEQ ID NO: 1). Likewise, other Nluc variants may also be used in the invention, provided that they have substantially the same activity as and Nluc (SEQ ID NO: 2). As used herein, the term “substantially the same” when referring to the activity of two luciferases means that when reacting with the same substrate, the difference in signals generated by the two luciferases is less than 40%, less than 30%, less than 20%, less than 10% of the values of the lesser signal. For example, a luciferase with substantially the same activity as Gluc will react with CTZ to generate oxidized CTZ, and a luciferase with substantially the same activity as Nluc will react with f-CTZ to generate oxidized f-CTZ. In one aspect, the Gluc variant comprises a sequence that is identical to, or substantially identical to a subsequence or the full length sequence of SEQ ID NO:1. In one aspect, the Nluc variant comprises a sequence that is identical to, or substantially identical to a subsequence or the full length sequence of SEQ ID NO:2. An amino acid sequence that is substantially identical to a reference sequence (e.g., SEQ ID NO: 1 or SEQ ID NO: 2) when it has at least 70%, at least 80%, or at least 90% sequence identity with the corresponding portion of the reference sequence. In some embodiments, the Gluc variant has a sequence that differs from SEQ ID NO:1 by no more than one, no more than two, no more than three, no more than four, no more than five, no more than eight, no more than 10 amino acid residues and shares substantially the same activity as Gluc (SEQ ID NO: 1). In some embodiments, the Nluc variant has a sequence that differs from SEQ ID NO:2 by no more than one, no more than two, no more than three, no more than four, no more than five, no more than eight, no more than 10 amino acid residues and has substantially the same activity as the Nluc (SEQ ID NO: 2).

### Substrates

A luciferase polypeptide and its specific substrate is referred to herein as an orthogonal enzyme/substrate pair or orthogonal pair in this disclosure. In one embodiment, the orthogonal substrate for Gluc is CTZ. In one embodiment, the orthogonal substrate for Nluc is f-CTZ, which has a chemical structure (structure I):

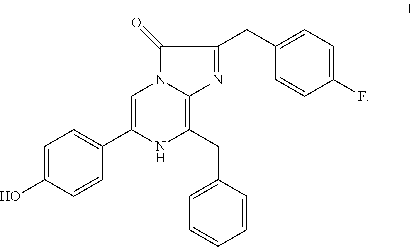 [SMILES: O=c1c(Cc2ccc(F)cc2)nc2c(Cc3ccccc3)[nH]c(-c3ccc(O)cc3)cn1-2]

### Substrate Stock Buffer

Luciferase substrates are typically prepared in solvents or stock buffers. For substrates that are not water-soluble such as, CTZ and f-CTZ, various organic solvents may be used. Non-limiting examples of organic solvents that can be used to prepare the substrates disclosed herein include ethanol, propylene glycol, methanol, DMSO, and mixtures thereof at different ratios. In one embodiment the organic solvent contains 50% v/v ethanol and 50% v/v propylene glycol. Substrates in stock buffers with this formulation have demonstrated the optimal solubility, stability and signal intensity.

### Luciferase-Substrate Reaction Buffer

In some embodiments, substrates (e.g., CTZ and f-CTZ) used herein may be sensitive to oxidation. Various approaches can be used to prevent oxidation of the substrate and to ensure optimal signal development. These approaches include, but not limited to, adding one or more anti-oxidants (e.g., sodium ascorbate) to the luciferase substrate reaction buffer; adding an agent (e.g., PEG3350) that can increase the viscosity of the substrate buffer; and/or maintaining the substrate buffer at a pH within a range that is both suitable for luciferase substrate reaction and maintaining the stability of the substrate. In some embodiments, the pH is in the range from pH 7.5 to 8.5, e.g., pH 8. In one particular embodiment, the reaction buffer has the following components:
50 mM Tris-HCl, pH=8.0
0.5M NaCl
0.1% Tween® 20 (or polysorbate 20)
0.1M Sodium Ascorbate
1% (w/v) PEG 3350

### Affinity Reagents

An affinity reagent can be used to detect the presence or absence of an 3′-O-reversible terminator deoxyribonucleotide (“NLRT”) incorporated at the 3′ end of a nucleic acid (the 3′ end of a GDS). The 3′-O-reversible terminator deoxyribonucleotides may comprise a nucleobase selected from the group consisting of adenine (A), cytosine (C), guanine (G), thymine (T), and analogs thereof. An affinity agent can specifically bind an NLRT based on a structural feature of the incorporated NLRT, for example, the affinity reagent may specifically bind to 3′-O-reversible terminator deoxyribonucleotides having, a particular base and/or particular reversible blocking group.

In one approach, the affinity reagent binds specifically to the nucleobase and distinguishes among different bases (A, T, G, C) in part based on the presence or absence of a 3′-OH group. In this approach the affinity reagent distinguishes a nucleotide at the 3′ end of a GDS with a 3′-OH from incorporated nucleotides interior to the GDS (not at the 3′ end). In some cases the affinity reagent recognizes a specific nucleobase and also distinguishes between the presence or absence of a 3′-OH groups, which allows identification of an incorporated NLRT as a 3′ terminal nucleotide with a particular nucleobase.

In one approach the affinity reagent recognizes an epitope comprising the blocking group but does not distinguish between bases. For example, given four RT blocking groups [A. azidomethyl, B. 2-(cyanoethoxy)methyl, C. 3′-O-(2-nitrobenzyl), and D. 3′-O-allyl] affinity reagents can be produced that distinguish the four blocking groups. For illustration, given the deoxyguanine analogs labeled A to D below, an affinity reagent can be selected that recognizes only one, but not the other three, NLRTs.
A. 3′-O-azidomethyl-2′-deoxyguanine
B. 3′-O-2-(cyanoethoxy)methyl-2′-deoxyguanine
C. 3′-O-(2-nitrobenzyl)-2′-deoxyguanine
D. 3′-O-allyl-2′-deoxyguanine

Examples of affinity reagents herein include antibodies (including binding fragments of antibodies, single chain antibodies, bispecific antibodies), aptamers, knottins, affimers, guanine nucleotide binding proteins (G-proteins), or any other known agent that binds an incorporated NLRT with a suitable specificity and affinity. Non-limiting examples of affinity reagents, including antibodies, are described in WO2020097607, the entire disclosure of which is herein incorporated by reference.

### Antibodies as Affinity Reagents

In some embodiments, the affinity reagents are antibodies that can specifically bind to NLRTs and distinguish NLRTs comprising different nucleobases. As used herein, “antibody” means an immunoglobulin molecule or composition (monoclonal and polyclonal antibodies), as well as genetically engineered forms such as chimeric, humanized and human antibodies, heteroconjugate antibodies (such as bispecific antibodies), and antibody fragments. The antibody may be from recombinant sources and/or produced in animals, including without limitation transgenic animals.

The term “antibody” includes “antibody fragments,” including without limitation Fab, Fab′, F(ab′)2, scFv, dsFv, ds-scFv, dimers, minibodies, nanobodies diabodies, and multimers thereof and bispecific antibody fragments. Antibodies can be fragmented using conventional techniques. For example, F(ab′)2 fragments can be generated by treating an antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques. The antibodies can be in any useful isotype, including IgM and IgG, such as IgG1, IgG2, IgG3 and IgG4. In some embodiments, the affinity reagents are minibodies. Minibodies are engineered antibody constructs comprised of the variable heavy (VH) and variable light (VL) chain domains of a native antibody fused to the hinge region and to the CH₃ domain of the immunoglobulin molecule. Minibodies are thus small versions of whole antibodies encoded in a single protein chain which retain the antigen binding region, the CH₃ domain to permit assembly into a bivalent molecule and the antibody hinge to accommodate dimerization by disulfide linkages. A single domain antibody (sdAb) may also be used. A single domain antibody, or NANOBODY (Ablynx), is an approximately antibody fragment with a single monomeric variable antibody domain. Single domain antibodies bind selectively to specific antigens and are smaller (MW 12-15 kDa) than conventional antibodies.

### Aptamers as Affinity Reagents

In some embodiments, the affinity reagents are aptamers that can specifically bind to NLRTs and distinguish NLRTs having different nucleobases. An aptamer is an oligonucleotide or peptide molecule that binds to a specific target molecule. Aptamers can be classified as: (a) DNA or RNA or XNA aptamers, which consist of (usually short) strands of oligonucleotides; and (b) peptide aptamers, which consist of one (or more) short variable peptide domains, attached at both ends to a protein scaffold.

Nucleic acid aptamers are nucleic acid species that have been engineered through repeated rounds of in vitro selection or equivalently, SELEX (systematic evolution of ligands by exponential enrichment) to bind to various molecular targets, such as NLRTs. For example, aptamers with affinity for a target NLRT can be selected from a large oligonucleotide library through SELEX, an iterative process in which non-binding aptamers are discarded and aptamers binding to the proposed target are expanded. Initial positive selection rounds are sometimes followed by negative selection. This improves the selectivity of the resulting aptamer candidates. In this process, the target NLRT is immobilized to an affinity column. The aptamer library is applied and allowed to bind. Weak binders are washed away and bound aptamers are eluted and amplified using PCR. Then the pool of amplified aptamers are reapplied to the targets. The process is repeated multiple times under increasing stringency until aptamers of the desired selectivity and affinity are obtained. See Jayasena, et al., Clinical Chemistry 45:1628-1650, 1999. Peptide aptamer selection can be made using different systems, including the yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. See Reverdatto et al., 2015, Curr. Top. Med. Chem. 15:1082-1101.

### Affimers As Affinity Reagents

In some embodiments, the affinity reagents are affimers that can specifically bind to NLRTs and distinguish NLRTs having different nucleobases. Affimers are small (12-14 kDa), highly stable proteins that bind their target molecules with specificity and affinity similar to that of antibodies. These proteins share the common tertiary structure of an alpha-helix lying on top of an anti-parallel beta-sheet. Affimer proteins display two peptide loops and an N-terminal sequence that can all be randomized to bind to desired target proteins with high affinity and specificity in a similar manner to monoclonal antibodies. Stabilization of the two peptides by the protein scaffold constrains the possible conformations that the peptides can take, increasing the binding affinity and specificity compared to libraries of free peptides.

Affimers specific for a NLRT can be selected by the use of phage display libraries that are screened to identify an Affimer protein with high-specificity binding to the target NLRT and high binding affinities (such as in the nM range). Many different labels, tags and fusion proteins, such as fluorophores, have been conjugated to Affimer proteins for use in various applications. See U.S. Pat. Nos. 8,481,491, 8,063,019, and WO 2009/136182, which are incorporated herein by reference. See also Crawford et al., Brief Funct. Genomic Proteomic, 2:72-79, 2003.

### Knottins as Affinity Reagents

In some embodiments the affinity reagents are knottins that can specifically bind to NLRTs and distinguish NLRTs having different nucleobases. “Knottin” or “inhibitor cystine knot” (ICK) is a protein structural motif containing three disulfide bridges. Along with the sections of polypeptide between them, two disulfides form a loop through which the third disulfide bond (linking the third and sixth cysteine in the sequence) passes, forming a knot. New binding epitopes can be introduced into natural knottins using protein engineering, and knottins have been engineered to target a broad range of targets. One approach to production of knottins that are specific for NLRTs is to create and screen knottin libraries using yeast surface display and fluorescence-activated cell sorting. For information regarding production of knottins with selectivity and high affinity for a target NLRT and labeling such knottins, see Kintzing and Cochran, Curr. Opin. Chem. Biol. 34:143-150, 2016; Moore et al., Drug Discovery Today: Technologies 9(1):e3-e11, 2012; and Moore and Cochran, Meth. Enzymol. 503:223-51, 2012.

### Luciferase-Coupled Affinity Reagents

A luciferase as disclosed herein can be coupled to an affinity reagent as described below. In the presence of its orthogonal substrate, the luciferase-coupled affinity reagent binds to a primer extension product and produces a detectable luminescent signal. In some embodiments, a luciferase-coupled affinity reagent disclosed herein is an affinity reagent that is coupled to a luciferase either directly or indirectly (e.g. via a linker), covalently or non-covalently. In some embodiments, the luciferase-coupled affinity reagent is a fusion protein comprising a luciferase polypeptide and an affinity reagent. In one approach, the luciferase polypeptide is directly fused to an affinity reagent that is a polypeptide (“protein affinity reagent”). In another approach the luciferase polypeptide is covalently linked to the affinity reagent. In yet another approach the luciferase polypeptide is bound to the affinity reagent noncovalently. For example, the affinity reagent may comprise a biotin group, which binds to a streptavidin group on the luciferase polypeptide.

### Affinity Reagents Covalently Linked to a Luciferase Polypeptide

In some embodiments, the affinity reagent used herein is covalently linked to the luciferase polypeptide through a linker. Protein affinity reagents (e.g., antibodies) can be covalently modified in many ways to suit the purpose of a particular assay. The functional groups on a protein (e.g., an antibody) that are used for conjugation include one of three targets: (1) Primary amines (—NH₂); (2) Sulfhydryl groups (—SH); and Carbohydrates (sugars). Primary amines occur on lysine residues and the N-terminus of each polypeptide chain and they are numerous and distributed over the entire antibody. Sulfhydryl groups occur on cysteine residues and exist as disulfide bonds that stabilize the whole-molecule structure. Hinge-region disulfides can be selectively reduced to make free sulfhydryls available for targeted labeling. Carbohydrates (sugars) may occur primarily in the Fc region of antibodies (IgG). In some cases, component sugars in these polysaccharide moieties that contain cis-diols can also be oxidized to create active aldehydes (—CHO) for coupling. The various chemical approaches for antibody labeling are summarized in US-2018-0223358-A1, the entire content of which is herein incorporated by reference.

In some embodiments, the affinity reagent is coupled to a luciferase (e.g., Nluc) via an SMCC linker. SMCC has the following structure (structure II):

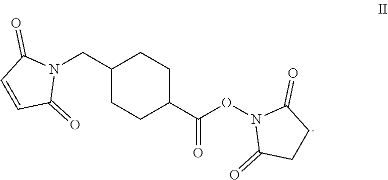 [SMILES: O=C(ON1C(=O)CCC1=O)C1CCC(CN2C(=O)C=CC2=O)CC1]

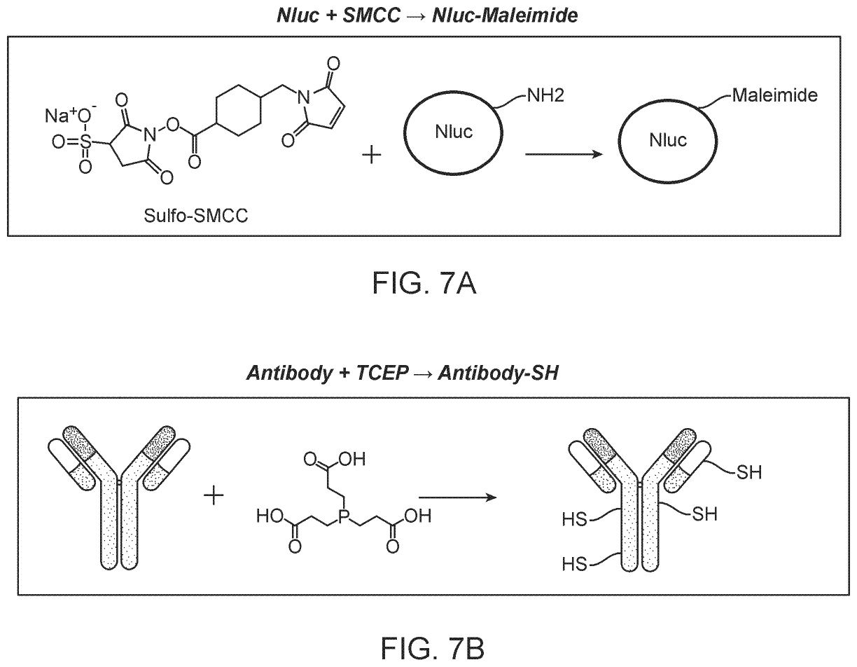

In various locations of this disclosure, SMCC is also referred to as the SMCC linker when connecting two molecules (e.g., an SMCC linker connecting a luciferses and an antibody). In some embodiments, the affinity reagent is an NLRT antibody, which is treated with a reducing agent (e.g., Tris (2-carboxyethyl) phosphine hydrochloride (TCEP)) to open the hinge-region disulfides in the antibody and generate sulfhydryl groups (—SH). In some embodiments, the antibody is treated with a reducing agent before conjugation with the luciferase polypeptide, and the molar ratio of the antibody to the reducing agent ranges 1:1 to 1:5. The coupling of the antibody to the luciferase is shown in FIGS. 7A and 7B. An SMCC is linked to the antibody via a covalent bond formed between a —SH group on the antibody formed as above and a maleimide group on the SMCC linker. The same SMCC molecule is also linked to a luciferase via a covalent bond, and the covalent bond is formed between the —NHS group on the SMCC linker and a primary amine group on the luciferase. In some embodiments, the affinity reagent is coupled to the Nluc via an SMCC linker. In some embodiments, the affinity reagent is coupled to the Gluc via an SMCC.

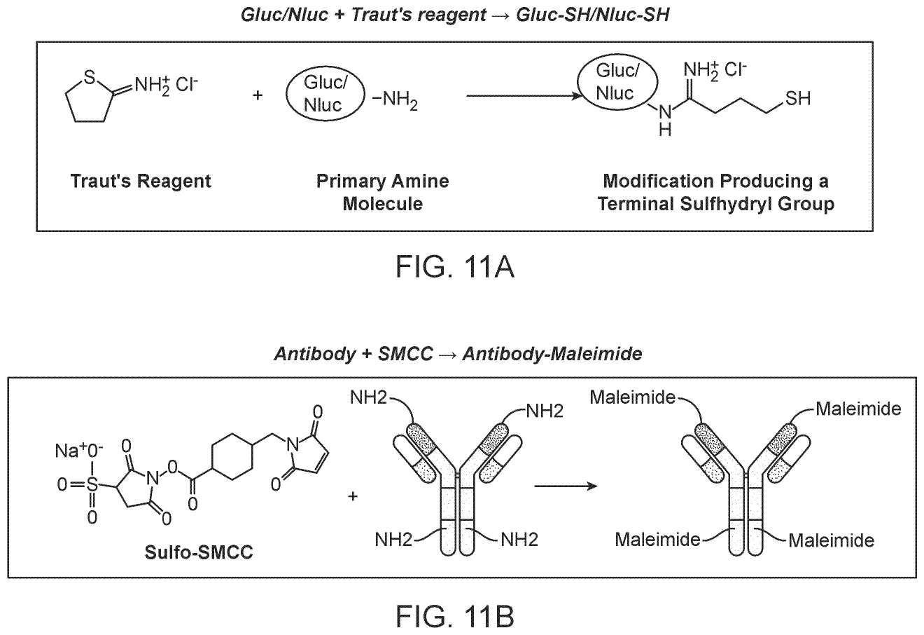

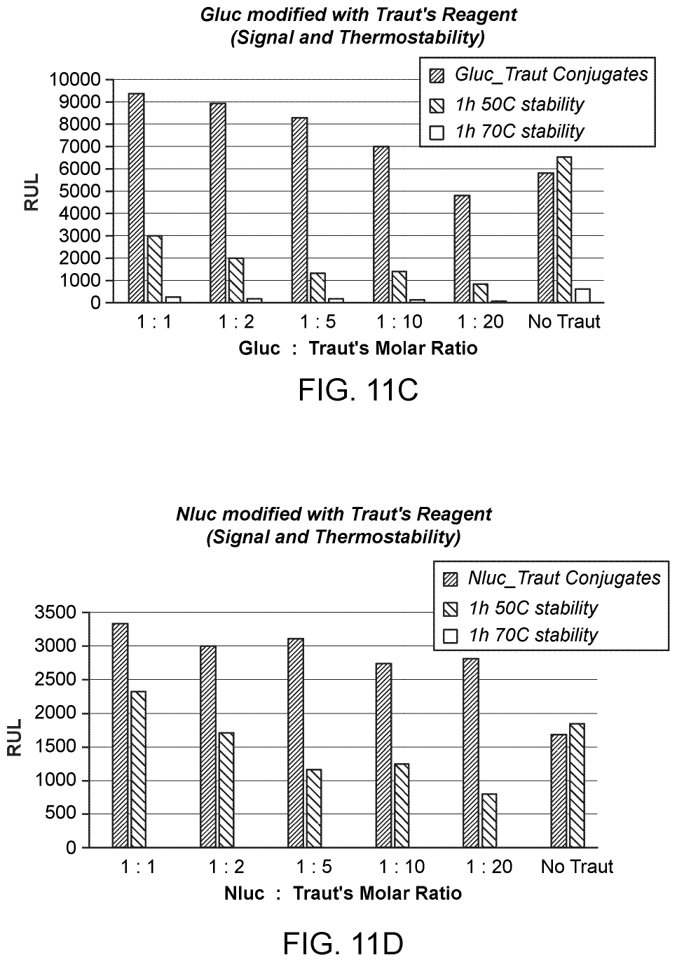

In some embodiments, the affinity reagent is coupled to a luciferase polypeptide, for example, Gluc or Nluc, which has been treated with a Traut's reagent. In some embodiments, the affinity reagent is coupled to the luciferase polypeptide via i) a covalent bond between a —SH group on the luciferase and a maleimide group on the SMCC, and ii) a covalent bond between the —NHS group of the SMCC and the —NH2 group on the affinity reagent. As shown in FIGS. 11C and 11D, treating the luciferase (Gluc or Nluc) with the Traut's reagent significantly increase the signal intensity and the stability of the luciferase—an increase of 60% for Gluc and increase of 100% for Nluc.

In some embodiments, the molar ratio between the luciferase polypeptide and the antibody ranges from 1:3 to 1:20, e.g., about 1:10.

### Affinity Reagents Fused to a Luciferase Polypeptide

In some embodiments, the luciferase polypeptide is fused to a protein affinity reagent to form a recombinant luciferase-affinity reagent fusion protein. In some embodiments, the protein affinity reagent is an antibody or an antibody fragment, e.g., a single-chain Fv fragment (ScFv). A variety of approaches are known in the art for linking a luciferase polypeptide sequence to a target protein. See, e.g., Wouters et al., 2020, “Bioluminescent Antibodies through Photoconjugation of Protein G-Luciferase Fusion Proteins” Bioconjugate Chem. 31, 3, 656-662.

### Affinity Reagents Coupled to a Luciferase Polypeptide Through Non-Covalent Means

In some embodiments, the affinity reagent is coupled to the luciferase via a non-covalent means, e.g., through the interaction of two binding partners. Non-limiting examples of binding partners include streptavidin and biotin, antigen and antibody, nitriloacetic acid nickel complex and histidine tag. For example, the luciferase may be conjugated to a biotin or a streptavidin, the affinity reagent may be conjugated to a streptavidin or a biotin, and the luciferase polypeptide is then coupled to the affinity reagent through the binding of the biotin and the streptavidin on both molecules.

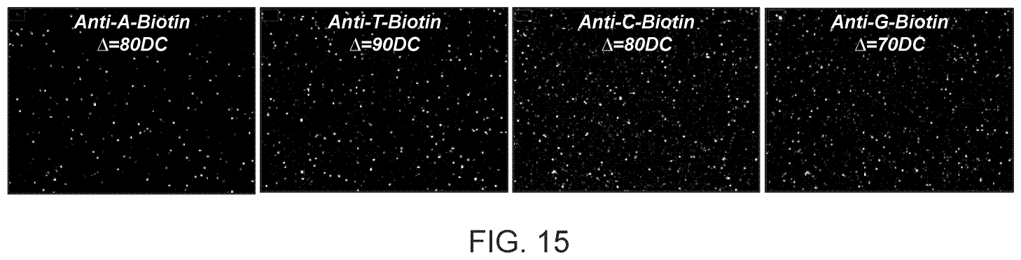

The ratio between a binding partner to the affinity reagent may vary. In some approaches, the ratio of the affinity reagent to one of the two protein binding partners may be in a range from 1:5 to 1:20, e.g., from 1:6 to 1:15, from 1:8 to 1:12, or about 1:10. In one example, labeling an antibody with biotin using a ratio of antibody:biotin at 1:10 produced signal that is sufficiently high for the one color sequencing scheme. See FIG. 15.

### Luciferase-Coupled Affinity Reagent Sets

The term “a luciferase-coupled affinity reagent set” refers to a set of two or more affinity reagents, each specifically recognizing one of the NLRTs. In some approaches the method and compositions disclosed herein use a set of luciferase-coupled affinity reagents, in which at least two affinity reagents are coupled with two different luciferase polypeptides. The two different luciferase polypeptides used in the set do not have significant cross-substrate reactivity.

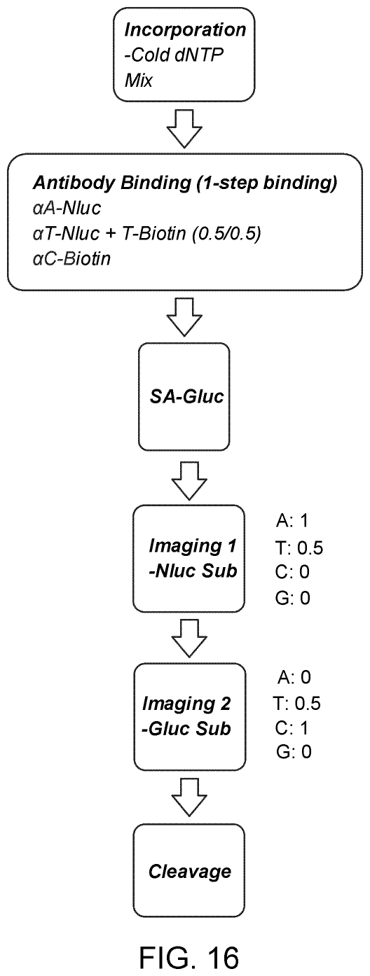

In some approaches, the set of luciferase-coupled affinity reagents comprise four luciferase-coupled affinity reagents: a first affinity reagent that is coupled to the first luciferase polypeptide, a second affinity reagent that is coupled to the second luciferase polypeptide, a third affinity reagent that is coupled to both the first and the second luciferase polypeptide, and a fourth affinity reagent that is coupled to neither the first nor the second luciferase polypeptide. The first luciferase polypeptide is different from the second luciferase polypeptide. In some embodiments, the third affinity reagent is covalently linked to the first luciferase polypeptide but is noncovalently coupled to the second luciferase polypeptide. For example, as shown in FIG. 16, a third affinity reagent may be covalently linked to a Nluc via an SMCC linker and also covalently linked to a biotin. The biotin group conjugated to the third affinity reagent can bind to a streptavidin conjugated to a Gluc so that the third affinity reagent can be coupled to both luciferase polypeptides.

In some approaches the first luciferase polypeptide is Gluc and the second luciferase polypeptide is Nluc. In one particular embodiment the Gluc has a sequence of SEQ ID NO: 1 and the Nluc has a sequence of SEQ ID NO: 2.

### Luciferase-Coupled Binding Pair

In some embodiments, a luciferase is recruited to a NLRT incorporated to the end of the GDS through a binding pair. The binding pair consists of two binding partners, one conjugated to the luciferase and the other conjugated to the NLRT. Non-limiting examples of the binding pairs include streptavidin and biotin, antigen and antibody, nitriloacetic acid nickel complex and histidine tag, and the like. For example, the luciferase may be conjugated to a streptavidin, the NLRT may be conjugated to a biotin, and the luciferase polypeptide is recruited to the NLRT through the binding of the biotin and the streptavidin.

In some embodiments, the sequencing reaction includes a NLRT set. The NLRT set includes a first, second, third and fourth NLRTs. At least two luciferases including a first luciferase and a second luciferase are used in the reaction. The first NLRTs are bound to the first luciferase to through a first binding pair, one binding partner conjugated to the first luciferase and the other binding partner conjugated to the first NLRTs. The second NLRTs are bound to the second luciferase through a second binding pair, one binding partner conjugated to the second luciferase and the other binding partner conjugated to the second NLRT. Some of the third NLRTs are conjugated to a binding partner of the first binding pair, which are bound to the first luciferase through a first binding pair. Some of the third NLRTs are conjugated to a binding partner of the second binding pair, which are bound to the second luciferase through a second binding pair.

In some embodiments, the first luciferase is Gluc, and the second luciferase is Nluc. In some embodiments, the first binding pair is a streptavidin and a biotin, and the second binding pair is an antigen and an antibody that specifically recognizes the antigen. In some embodiment, the antigen is digoxin and the antibody is an anti-digoxin antibody.

One illustrative example is in Example 5, in which 3′-azidomethyl-dATPs are conjugated to biotin, 3′-azidomethyl-dCTP are conjugated to digoxin, some of the 3′-azidomethyl-dUTP are conjugated to biotin, and some of the 3′-azidomethyl-dUTP are conjugated to digoxin. 3′-azidomethyl-dUTPs are conjugated to neither biotin nor digoxin. In a sequencing reaction, streptavidin-conjugated Gluc binds to the biotin-labeled 3′-azidomethyl-dATPs and biotin-labeled 3′-azidomethyl-dUTPs, and anti-digoxin antibody conjugated Nluc binds to digoxin-labeled 3′-azidomethyl-dCTPs and digoxin-labeled 3′-azidomethyl-dUTPs. The binding of Gluc and Nluc to the NLRTs can be detected by their respective orthogonal substrates.

### Immobilizing Affinity Reagents

Affinity reagents (e.g., luciferase-coupled affinity reagent) can be immobilized on a solid support (e.g., an array) on which a DNA template to be sequenced is immobilized. Various means can be used to achieve this purpose. In one approach, the affinity reagent binds directly to an NLRT at the end of the GDS. In one approach, the affinity reagent binds to an NRLT that has been modified to include a binding partner and said binding partner binds to the affinity reagent. In yet another approach, the affinity reagent binds to a binding partner and said binding partner binds to the NRLT. Other variations of immobilizing means can be readily appreciated by one of skilled in the art and are also encompassed within this disclosure.

### Kits

The luciferase-coupled affinity reagents in the set can be provided in kit form, as a mixture or in separate containers. A kit disclosed herein may include luciferase-coupled affinity reagents or affinity reagent sets as described above. The kit may additionally comprise NLRTs and NLRT sets. For example, kits may include, without limitation (a) a NLRT or NLRT set that includes one, two, three, four or more different individual NLRTs; and (b) a corresponding affinity reagent or affinity reagent set that includes one, two, three, four or more affinity reagents, each of which is specific for one of the NLRTs, and at least two of the affinity reagents are coupled to two luciferase polypeptides. The kit may further include packaging materials and/or instructions for use. The two luciferase polypeptides do not have significant cross-substrate reactivity. In some embodiments, the kit further comprise the orthogonal substrate for each luciferase polypeptides, e.g., the CTZ for the Gluc-coupled affinity reagent and f-CTZ for the Nluc-coupled affinity reagent. In some embodiments, the kit further comprises an antioxidant to prevent the oxidation of the first, second, or both substrates. In some embodiments, the kit further comprise a Traut's reagent.

In some embodiments the kit comprises secondary affinity reagents bind to the first or second affinity reagents. In some embodiments, the first or second affinity reagent is coupled to a luciferase through binding to a secondary affinity reagent that is covalently linked to the luciferase polypeptide. In some embodiments the first and/or second affinity reagents are antibodies.

Production of the Affinity Reagents that are Covalently Linked to Luciferase Polypeptides

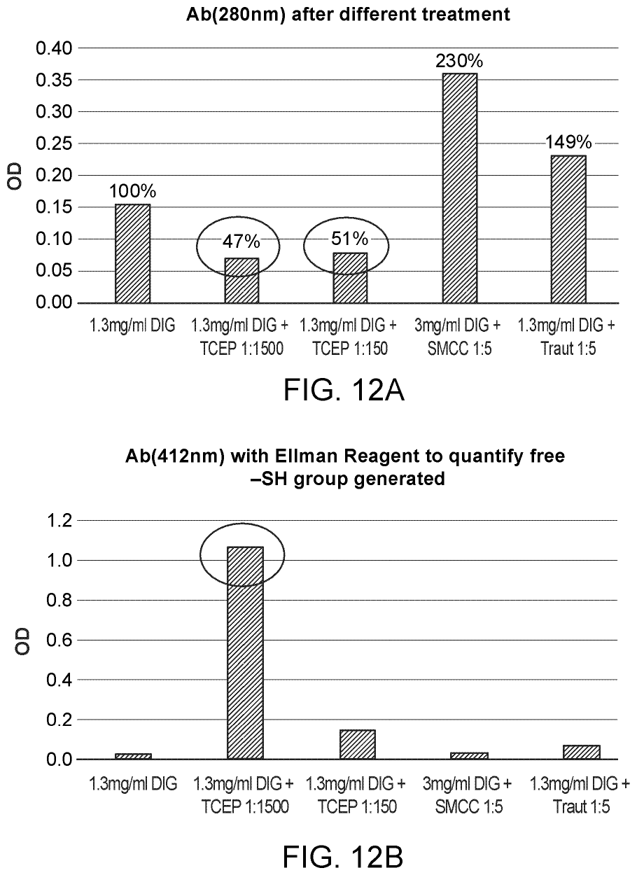

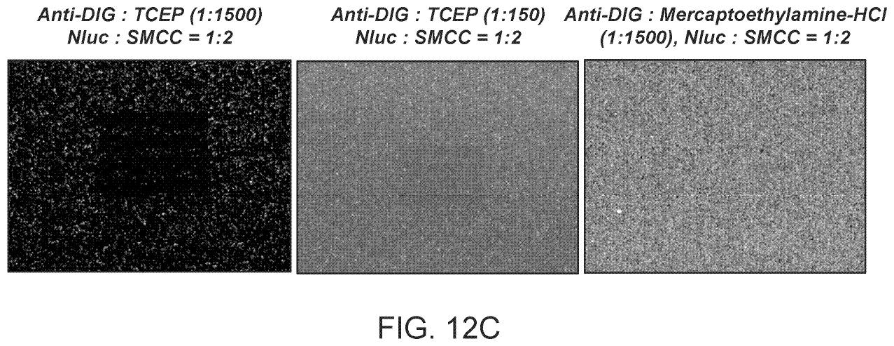

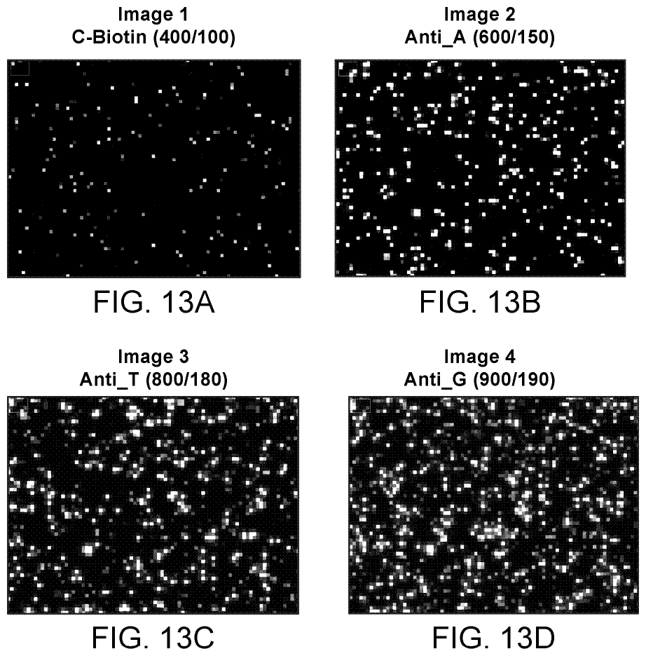

In one embodiment, the affinity reagent is an antibody that recognizes one of the NLRTs. An antibody can be covalently conjugated to the luciferase polypeptide through the various means. In some approaches, the antibody can be covalently conjugated to the luciferase polypeptide through an SMCC linker. In this approach, the method of producing the luciferase-affinity reagent conjugate may comprise treating the affinity reagent (e.g., a NLRT antibody) with a reducing agent to generate free —SH groups on the antibody. In some embodiments, the reducing agent is Tris (2-carboxyethyl) phosphine hydrochloride (TCEP). It is desirable to keep the ratio of the reducing agent and the linker (SMCC, Traut's) at an optimal ratio in order to achieve high signal intensity from the luciferase affinity reagent conjugate. In some embodiments the ratio of the reducing agent to the linker is in a range from 1:150 to 1:3000, e.g., from 1:200 to 1:2000, or from 1:1000 or 1:1800. In one particular embodiment maintaining the ratio of the reducing agent to the linker at 1:1500 was able to achieve the highest SH labeling degree and highest signal from the sequencer imager. See FIG. 12A-12C. An antibody treated with the reducing agent is then linked to an SMCC via a covalent bond formed between the —SH group on the antibody and the maleimide group on the SMCC; the SMCC is also linked to a luciferase polypeptide via a covalent bond formed between the —NHS group on the SMCC and the primary amine (—NH2) group on the luciferase, thereby forming an antibody-luciferase polypeptide conjugate. In some embodiments, the antibody is coupled to the Nluc via the SMCC.

In some approaches the luciferase polypeptide is first treated with a thiolation agent before conjugating with a suitable linker, for example, an SMCC. Treatment with the thiolation agent produces free sulfhydryl groups on the luciferase polypeptide, and introduction of these sulfhydryl groups can boost the luciferase's enzymatic activity. In some embodiments, the thiolation agent is a cyclic thioimidate compound. In some embodiments the thiolation agent is 2-iminothiolane, also commonly known as the Traut's agent.

In one illustrative example, as shown in FIG. 11A, the luciferase polypeptide (e.g., Gluc) is treated with the Traut's reagent to produce a free —SH group on the luciferase. The NLRT antibody is treated with an SMCC linker such that a covalent bond is formed between the primary amine on antibody and the —NHS group on the SMCC, as illustrated in FIG. 11B. The reaction (1) between the Traut's reagent and the luciferase and the reaction (2) between the antibody and SMCC may be performed in any order or simultaneously. The luciferase treated with the Traut's agent and the antibody treated with the SMCC are mixed to form a covalent bond between the —SH group on the luciferase polypeptide and the maleimide group on the SMCC, forming the luciferase antibody conjugate. This method is especially useful for production of Gluc-conjugated NLRT antibody, when efforts of conjugating Gluc with NLRT with an SMCC linker without the Traut' agent treatment were not successful. See Example 3.

### Sequencing Process in General

The methods and compositions disclosed herein can be used in combination with a number of sequence methods including sequencing-by-synthesis (SBS) using unlabeled reversible terminator nucleotides. SBS methods are well known including, but not limited to, methods described in references cited herein, each of which is incorporated by reference for all purposes. Typically SBS determines sequence of a single-stranded nucleic acid template immobilized at a position on a surface. As is known to the reader of ordinary skill in the art, usually there are many copies of the template at a position on the surface. For illustration and not limitation, the template copies are most often produced using DNA nanoball (DNB) methods or bridge PCR methods. DNB methods result in a single stranded concatemer with many copies of the template (e.g., genomic DNA sequences and adjacent primer binding sites). Bridge PCR methods result in a clonal cluster of template molecules (e.g., genomic DNA sequences flanked by adaptors which may serve as primer binding sites). In bridge PCR both strands of the template nucleic acid may be present, as separate single strands. It will be understood that references herein to a “template” nucleic acid (i.e., singular grammatical form), or equivalent terms, also refers to a plurality of copies of a template at a given position on a substrate. It will also be recognized that, although reference may be made herein to determining sequence of a template nucleic acid or template nucleic acid sequence (i.e., singular grammatical form), it is contemplated the methods of the invention are carried out using arrays comprising a plurality (often hundreds of millions) of positions containing one or a plurality of template nucleic acid molecules.

### Two Label, One Color Sequencing

The present method of using two differently labeled affinity reagents allows simultaneous delivery of different NLRT affinity reagents, each recognizing an NLRT having different nucleobases. In some embodiments, two differently labeled affinity reagents refer to two luciferase polypeptides; not only the two luciferase polypeptides each can generate high intensity luminescent signals when reacting with their respective orthogonal substrate, they also do not have significant cross-substrate reactivity cross talk. Thus, when coupled with two affinity reagents, each recognizing a NLRT having a different nucleobase, at least the two different nucleobases can be distinguished.

In one embodiment, the two-label, one-color sequencing method comprises: providing a plurality of nucleic acid templates each comprising a primer binding site and, adjacent to the primer binding site, a target nucleic acid sequence; performing sequencing reactions on the plurality of different nucleic acid templates by hybridizing a primer to the primer binding site and extending individual primers by one nucleotide per cycle in one or more cycles of sequencing-by-synthesis using a set of NLRTs and a corresponding set of affinity reagents, e.g.: (i) first NLRTs and first affinity reagents that specifically bind to the first NLRTs and that is coupled to a first luciferase polypeptide; (ii) second NLRTs and second affinity reagents that specifically bind to the second NLRTs and that is coupled to a second luciferase polypeptide; (iii) third NLRTs and third affinity reagents that specifically bind to the third NLRTs and that is coupled to both the first luciferase polypeptide and the second luciferase polypeptide; and (iv) fourth NLRTs and fourth affinity reagents that specifically bind to the fourth NLRTs and that are unlabeled (i.e., coupled to neither the first nor the second luciferase polypeptide). In each cycle of sequencing-by-synthesis, the method comprises 1) contacting the array with the set of NLRTs and the set of NLRTs affinity reagents, 2) contacting the array with the first substrate, 3) detecting the first luminescent signal generated by the first affinity reagent cleaving the first substrate; 4) removing the first substrate; 5) contacting the array with the second substrate; 6) detecting the second luminescent signal generated by the second affinity reagent cleaving the second substrate; 7) determining the identities of NLRTs at the detection positions by detecting the presence and intensity (or absence) of the label to determine the target nucleic acid sequences.

In some embodiments, the first luciferase and second luciferase are added to a sequencing flow cell in one mixture and the first substrate is added. The first luciferase reacts with the first substrate and produces a first signal. After the detection of the first signal, the first substrate is washed away from the flow cell, and the second substrate is then added. The second luciferase reacts with the second substrate and produces a second signal. The second signal is also detected. The type of NLRT at each position of the flow cell is then determined based on the presence or absence of the first and second signal.

In some embodiments, the first luciferase and second luciferase are added sequentially. In some cases, the first luciferase and the first substrate are added to the flow cell to produce a first signal. After detection of the first signal, the second luciferase and second substrate are added to the flow cell. The second signal is produced and detected.

Optionally, before contacting the array with the second substrate and/or the second luciferase, a selective deactivation agent (as further discussed below) is added to the array or sequencing flow cell, and the agent is added in an amount that can selectively deactivate the first luciferase but not the second luciferase.

In one embodiment, the first luciferase is Gluc, the first substrate is CTZ, the second luciferase is Nluc, and the second substrate is f-CTZ.

In some embodiments, to further reduce sequencing error associated with the cross-substrate reactivity between the two luciferase polypeptides, after the signal from the first luciferase polypeptide (e.g., Gluc) is detected and recorded, a deactivation agent is applied to the sequencing array to deactivate the first luciferase polypeptide. Then a second substrate (the orthogonal substrate for the second luciferase) is added to the array followed detecting a second luminescent signal produced by the second luciferase (e.g., Nluc).

In preferred embodiments, the agent's deactivation activity is selective. That is to say, it deactivates the first luciferase polypeptide but does not substantially impair the enzymatic activity of the second luciferase polypeptide. As used herein, the term “deactivates,” when referring to a treatment to a luciferase, refers to that the luciferase's activity after the treatment, as measured by the luminescent signal generated by the luciferase polypeptide cleaving its orthogonal substrate, reduces at least 60%, at least 70%, at least 80% as compared to the activity of the luciferase before the treatment. The term “does not substantially impair” when referring to the effect of a treatment on the enzymatic activity of a luciferase, refers to that after the treatment, the luciferase polypeptide retains at least 60% at least 70% at least 80% at least 90% of its activity before the treatment

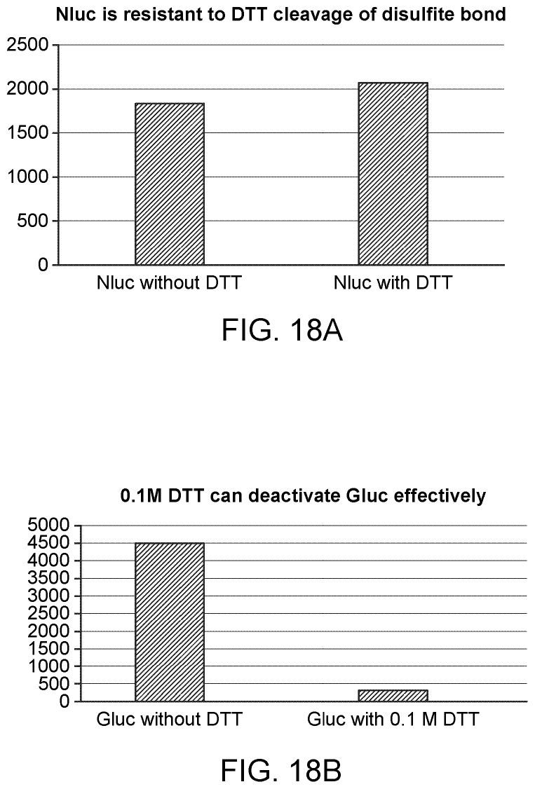

Selective deactivation agents that are suitable for the invention include, but not limited to, Dithiothreitol (DTT), beta-mercaptoethylamine, tris-carboxyethylphosphine (TCEP). The suitable concentrations of the agent (e.g., DTT) for selectively deactivating the first luciferease may range from 0.001M to 2M, e.g., from 0.01M to 0.5M, from 0.05M to 1M, 0.07M to 0.5M, or about 0.1M. In one exemplary assay, a Gluc luciferase almost lost enzymatic activity completely upon treatment with DTT, (FIG. 18B, which shows the signal reduced more than 95% after DTT treatment). In contrast, a Nluc was resistant to the DTT treatment (FIG. 18A, which shows that the DTT treatment did not impair the activity of the Nluc, the signal after the DTT treatment did not decrease but slightly increased).

Accordingly, in some embodiments, a selective deactivation agent (e.g., Dithiothreitol (DTT)) at a concentration that is effective at selectively deactivating the first luciferase (e.g., Gluc) is added to the wash buffer after the signal from the first luciferase (“the first luminescent signal”) is measured. The second substrate is then added and cleaved by the second luciferase. This produces a second luminescent signal, which can be detected.

### Reaction Mixtures

Various affinity reagents, NLRTs, DNA polymerase, and/or suitable buffers can be present as components of a reaction mixture for nucleic acid sequencing. Exemplary reaction mixtures include, but are not limited to, those containing (a) template nucleic acid; (b) polymerase; (c) oligonucleotide primer; (d) an NLRT, or a mixture of NLRTs having structurally different nucleobases; and (e) a mixture of luciferase coupled affinity reagents, each specifically recognize a NLRT as described above. Exemplary sequencing reaction mixtures of the invention include, but are not limited to, arrays comprising a plurality of different template nucleic acids immobilized at different locations on the array; (b) one or more DNA polymerases; (c) one or more oligonucleotide primers; (d) and one or a mixture of NLRTs; and (e) two affinity reagents each coupled to at least one of two luciferase polypeptides, as described above. Exemplary sequencing reaction mixtures of the invention include, but are not limited to, arrays comprising a plurality of different template nucleic acids immobilized at different locations on the array; (b) growing DNA strands (GDS); and (c) one or more luciferase-coupled affinity reagents (e.g., an affinity reagent set as described hereinabove). In some embodiments, the GDS comprises a 3′ NLRT. In some embodiments, the kit further comprises a deactivation agent that selectively deactives the first luciferase polypeptide, for example, DTT.

The invention will be further understood with reference to the following non-limiting experimental examples.

### Exemplary Embodiments

Embodiment 1. A method of corresponding positions on an array with differently labeled affinity reagents immobilized at the positions comprising
i) providing an array of positions wherein the array comprises first positions and second positions,
wherein a first affinity reagent is immobilized at least some of the first positions and a second affinity reagent is immobilized at least some of the second positions;
wherein the first affinity reagent is associated with a first luciferase polypeptide and the second affinity reagent is associated with a second luciferase polypeptide and
wherein the first luciferase polypeptide can react with a first substrate to generate a first luminescent signal,
wherein the second luciferase polypeptide can react with a second substrate to generate a second luminescent signal,
wherein the first substrate is orthogonal to the first luciferase polypeptide and the second substrate is orthogonal to the second luciferase polypeptide,
wherein the first luciferase polypeptide does not have significant cross-substrate reactivity with the second substrate and the second luciferase polypeptide does not have significant cross-substrate reactivity with the first substrate,
ii) contacting the array with the first substrate and detecting the first luminescent signal at positions at which the first affinity reagent is immobilized
iii) contacting the array with the second substrate and detecting the second luminescent signal at positions at which the second affinity reagent is immobilized,
iii) determining at a position the first affinity reagent is immobilized if the first luminescent signal is detected at said position, or
determining at a position the second affinity reagent is immobilized if the second luminescent signal is detected at said position.

Embodiment 2. The method of embodiment 1, wherein the first substrate is f-CTZ and the first luciferase polypeptide is Nluc.

Embodiment 3. The method of any one of embodiments 1-2, wherein the second substrate is f-CTZ and the second luciferase polypeptide is Nluc.

Embodiment 4. The method of any one of embodiments 1-3, wherein the first substrate is coeleterazine (CTZ), and the second substrate is f-CTZ.

Embodiment 5. The method of any one of embodiments 1-4, wherein the first luciferase polypeptide is a Gaussia luciferase polypeptide (Gluc) and the second luciferase polypeptide is a NanoZac luciferase polypeptide (Nluc).

Embodiment 6. The method of any one of embodiments 1-5, wherein the array comprises third positions and fourth positions, a third affinity reagent is immobilized at some of the third positions and a fourth affinity reagent is immobilized at at least some of the fourth positions,
wherein the third affinity reagent is associated with both the first luciferase polypeptide and the second luciferase polypeptide, and
wherein the fourth affinity reagent is associated with neither luciferase polypeptide,
wherein determining the first affinity reagent is immobilized at a position if the first luminescent signal is detected from the position,
determining the second affinity reagent is immobilized at a position if the second luminescent signal is detected from the position,
determining the third affinity reagent is immobilized at a position if both the first luminescent signal and the second luminescent signal are detected from the position,
determining the fourth affinity reagent is immobilized at a position if neither the first nor the second luminescent signal is detected from the position.

Embodiment 7. The method of any of preceding embodiments, wherein the first luminescent signal is detected before the second luminescent signal is detected, and
wherein a deactivation agent is added after detection of the first luminescence but before the detection of the second luminescent signal, and
wherein the deactivation agent selectively deactivates the first luciferase polypeptide.

Embodiment 8. The method of embodiment 7, wherein the deactivation agent is dithiothreitol (DTT).

Embodiment 9. The method of any one of embodiments 1-8, wherein the first or the second affinity reagent specifically binds to a 3′-O-reversible terminator deoxyribonucleotide comprising a nucleobase selected from the group consisting of adenine (A), cytosine (C), guanine (G), thymine (T), and analogs thereof.

Embodiment 10. The method of embodiment 9, the method further comprises identifying a type of 3′-O-reversible terminator deoxyribonucleotide associated with if the affinity reagent for the type of 3′-O-reversible terminator deoxyribonucleotide is determined to be immobilized on said position.

Embodiment 11. The method of embodiment 1, wherein the first luciferase polypeptide is conjugated to the first protein via a linker, and/or wherein the second luciferase polypeptide is conjugated to the second protein via a linker.

Embodiment 12. The method of embodiment 11, wherein the linker is an SMCC linker.

Embodiment 13. The method of any one of embodiments 1-12, wherein the first luciferase polypeptide comprises a —SH group linked to a maleimide group on the SMCC linker, and a —NHS group of the SMCC is linked to the —NH2 group on the first protein; and/or
wherein the second luciferase polypeptide comprises a —SH group linked to the maleimide group on the SMCC linker, and a —NHS group of the SMCC is linked to a —NH2 group on the second protein.

Embodiment 14. The method of embodiment 13, wherein the SH group on the first luciferase is generated by treating the first luciferase polypeptide with a cyclic thioimidate compound.

Embodiment 15. The method of embodiment 13, wherein the SH group on the second luciferase is generated by treating the second luciferase polypeptide with a cyclic thioimidate compound.

Embodiment 16. The method of any one of embodiments 14-15, wherein the cyclic thioimidate compound is 2-iminothiolane.

Embodiment 17. A kit for performing sequencing, the kit comprising:
(1) a first protein that is associated with a first luciferase polypeptide,
   wherein the first luciferase polypeptide can specifically react with a first substrate to generate a first luminescent signal,
(2) a second protein that is associated with a second luciferase polypeptide,
   wherein the second luciferase polypeptide can specifically react with a second substrate to generate a second luminescent signal,
(3) a first substrate, and
(4) a second substrate;
wherein the first luciferase polypeptide does not have significant cross-substrate reactivity with the second substrate and the second luciferase polypeptide does not have significant cross-substrate reactivity with the first substrate.

Embodiment 18. The kit of embodiment 17, further comprising a third protein and a fourth protein, wherein the third protein is associated with both the first and second luciferase polypeptide, and wherein the fourth protein is associated with neither the first nor the second luciferase polypeptide.

Embodiment 19. The kit of any one of embodiments 17-18, wherein the first luciferase polypeptide is Gluc and the second luciferase polypeptide is Nluc.

Embodiment 20. The kit of any one of embodiments 17-19, wherein the first substrate is coeleterazine (CTZ).

Embodiment 21. The kit of any one of embodiments 17-20, wherein the second substrate is f-CTZ, having the structure below:

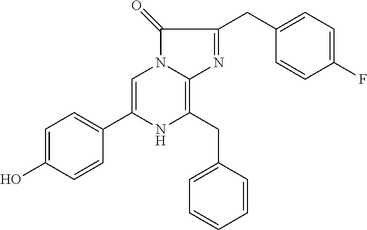 [SMILES: O=c1c(Cc2ccc(F)cc2)nc2c(Cc3ccccc3)[nH]c(-c3ccc(O)cc3)cn1-2]

Embodiment 22. The kit of any one of embodiments 17-21, wherein the kit further comprises a plurality of a 3′-O-reversible terminator deoxyribonucleotides, wherein each 3′-O-reversible terminator deoxyribonucleotide comprises a different nucleotide base.

Embodiment 23. The kit of any one of embodiments 17-22, wherein the first substrate or the second substrate is present in a stock buffer, wherein the stock buffer is selected from the group consisting of ethanol, propylene glycol, methanol, DMSO, and any combinations thereof.

Embodiment 24. The kit of embodiment 23, wherein the stock buffer is a mixture of 50% v/v ethanol with 50% v/v propylene glycol.

Embodiment 25. The kit of any one of embodiments 17-24, wherein the kit further comprises an antioxidant, wherein the antioxidant can prevent oxidation of the first substrate, the second substrate, or both the first and the second substrates.

Embodiment 26. A method of producing a luciferase-antibody conjugate, comprising:
(1) providing (i) an antibody that specifically recognizes a 3′-O-reversible terminator deoxyribonucleotides comprising a nucleobase that is selected from the group consisting of adenine (A), cytosine (C), guanine (G), thymine (T), and analogs thereof; and (ii) a luciferase polypeptide, and
(2) contacting the luciferase polypeptide with 2-iminothiolane under conditions that generate an —SH group on the luciferase polypeptide, thereby producing a luciferase polypeptide comprising an —SH group.
(3) contacting the antibody with an SMCC, wherein the —NHS group of the SMCC is linked to the —NH2 group on the antibody, thereby producing an SMCC-linked antibody having a maleimide group, and
(4) contacting the luciferase polypeptide comprising the —SH group with the SMCC-linked antibody under conditions suitable for protein conjugation thereby forming the luciferase-antibody conjugate.

Embodiment 27. The method of embodiment 26, wherein the luciferase polypeptide is a Gluc or a Nluc.

Embodiment 28. The method of any one of embodiments 26-27, wherein the luciferase polypeptide is conjugated to the antibody on a primary amine, a sulfhydryl group, or a carbohydrate.

Embodiment 29. The method of any one of embodiments 26-28, wherein the conjugation occurs in a reaction buffer, wherein the reaction buffer comprises Tris-HCL, NaCl, Tween 20, sodium ascorbate, and PEG 3350.

Embodiment 30. The method of any one of embodiments 26-29, wherein the molar ratio between the luciferase polypeptide and the antibody ranges from 1:3 to 1:20.

Embodiment 31. The method of any one of embodiments 26-30, wherein the antibody is treated with a reducing agent before conjugation with the luciferase polypeptide, wherein the molar ratio of the antibody to the reducing agent ranges between 1:1 and 1:5.

The method of embodiment 31, wherein the reducing agent is TCEP or

### EXAMPLES

### Example 1. Luciferase Selection

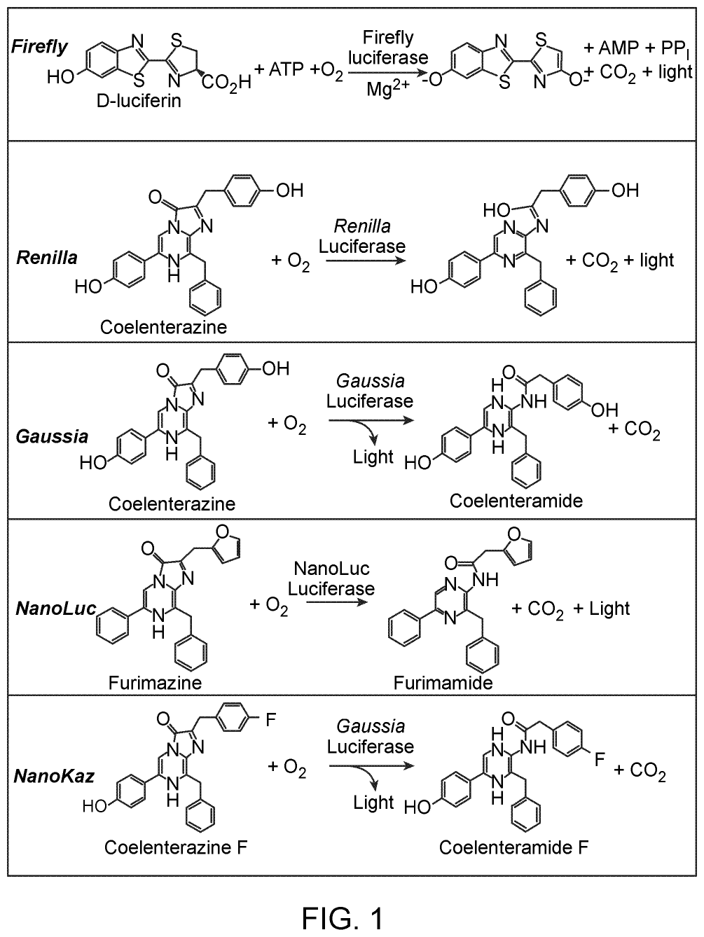

We have screened different luciferases on the market to select the most robust luciferases with high signal intensity and reducing agent good stability as the signaling labels for the bioluminescence-based sequencing chemistry. We evaluated a list of luciferases in terms of their signal intensity with the optimal substrate, protein size, signal lifetime, stability, etc., as shown in the Table 1 and FIG. 1.

In order to label 4 different NLRT antibodies to construct the one-color sequencing chemistry, two luciferase/substrate pairs were needed. The requirements to select these two luciferase/substrate pairs are 1) high signal intensity; 2) minimal cross-talk between the two luciferase/substrate pairs.

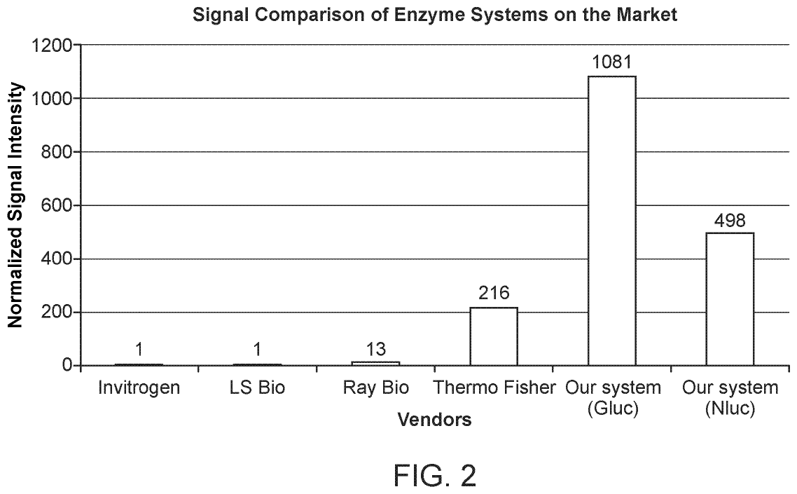

As a side-by-side comparison in signal intensity, the Gaussia luciferase (Gluc) mutant (Prolume Inc., J. Welsh, Biochemical and Biophysical Research Communications 389 (2009) 563-568, M43L, M110L mutant) selected in our system gave at least 5 fold signal enhancement compared to the best luciferase candidates from the competitive vendors, as shown FIG. 2. The other NanoZac luciferase (Nluc) mutant (Prolume Inc.) selected in our system shows half of the signal compared to the Gluc mutant. However, it was still multiple fold intensity higher than the rest of the counterparts. In this side-by-side comparison, each luciferase from different vendor reacted with its optimal substrate sold from the vendor. The integrated signal intensity from the first 10 sec was obtained from each luciferase/substrate pair to have the comparison.

The Gluc and Nluc were selected in our system not just because of their higher intensity compared to other luciferases, but minimal cross-talk was also demonstrated between these two luciferases with their own substrates. As shown in the FIG. 3 below, Gluc showed about 2% background signal when reacting with the Nluc substrate. While Nluc showed about 10% background signal when reacting with the Gluc substrate. Therefore, the two luciferases were selected as the bioluminescent signal labeling for our NLRT antibodies for the sequencing.

### Example 2. Luciferase Substrate Structure Screening and Formulation Development

Optimal substrate structures and buffer formulations were also developed for the two luciferases selected to show high signal intensity, good stability (long shelf life & on-board stability) and minimal cross-talk.

### 2.1 Substrate Structure Screening

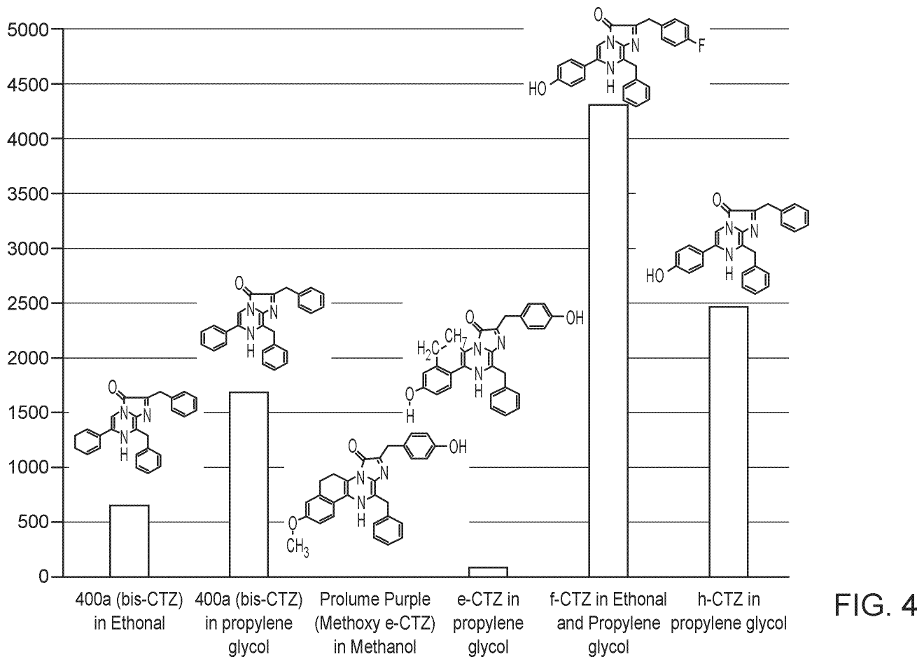

According to literature (J. Welsh, Biochemical and Biophysical Research Communications 389 (2009) 563-568), the optimal substrate for the selected Gluc mutant is coeleterazine (CTZ). We screened different analogs of CTZ with the Nluc mutant, and discovered that f-CTZ gave the highest signal intensity among the different CTZ analog structures, as showed in FIG. 4. Therefore, we identified that optimal substrate structure for the Nluc mutant is f-CTZ as shown below:

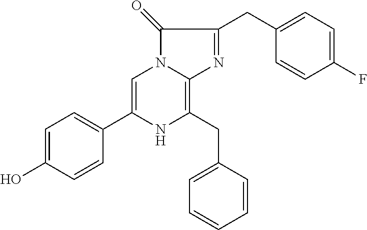 [SMILES: O=c1c(Cc2ccc(F)cc2)nc2c(Cc3ccccc3)[nH]c(-c3ccc(O)cc3)cn1-2]

### 2.2 Substrate Buffer Formulation Development

### 2.2.1 Substrate Stock Buffer Development

Since CTZ and f-CTZ were not water-soluble. An optimal organic solvent as substrate stock buffer was needed to obtain good solubility and good stability. Different organic solvents and mixes were tested, such as ethanol, propylene glycol, methonal, DMSO, and mixes of these solvents at different ratios. Results showed that 50% v/v ethanol with 50% v/v propylene glycol gave the best solubility, stability and signal intensity.

2.2.2 Substrate Reaction Buffer Development

Water-based substrate reaction buffer formulation was also developed to allow optimal reaction condition for the substrate with the luciferase. Since CTZ and f-CTZ are highly sensitive to oxidation, protecting CTZ and f-CTZ from oxygen before reaction with luciferase was critical. Sodium Ascorbate was selected as the effect anti-oxidant to help prevent CTZ and f-CTZ from oxidation. PEG3350 was also added into the formulation to increase the viscosity of the buffer and to slow down the oxidation by oxygen. Besides the anti-oxidants, pH is also another critical factor. Higher pH leads to easier oxidation. pH=8 has been found to be optimal pH for the luciferase/substrate reaction while keeping the substrate stable in buffer before reaction with all the anti-oxidants.

The optimal substrate reaction buffer formulation for both CTZ and f-CTZ was determined as below:
50 mM Tris-HCl, pH=8.0
0.5M NaCl
0.1% Tween 20
0.1M Sodium Ascorbate
1% (w/v) PEG 3350

To develop the substrate reaction buffer into a robust reagent kit for the bioluminescence-based sequencing platform, both on-board stability and long term shelf life have been evaluated. Since the typical run time for PE100 on the MGI DNBseq E series one-color sequencer is around 14 hours, when the whole reagent kit including the substrates in reaction buffer needs to stay at room temperature. We evaluated the room temperature stability for 24 hours (referred as on-board stability) for both CTZ and f-CTZ in the reaction buffer formulation listed above. Data shown in FIG. 5 indicated that the on-board stability of both CTZ and f-CTZ in this reaction buffer formation showed no significant difference as the fresh condition.

Long-term shelf life (long-term stability) was also evaluated for the substrate reaction buffer formulation. 300 uM substrate was diluted in the reaction buffer and stored at −20° C. One aliquot was thaw and tested against the fresh luciferase. Data at each time point was the relative signal intensity ratio compared to a fresh luciferase/substrate pair.

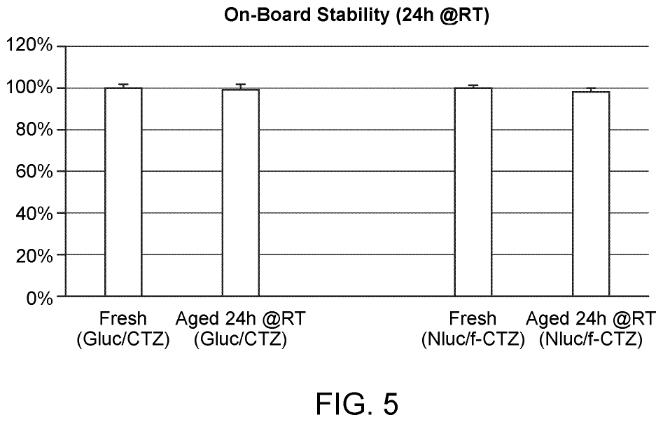

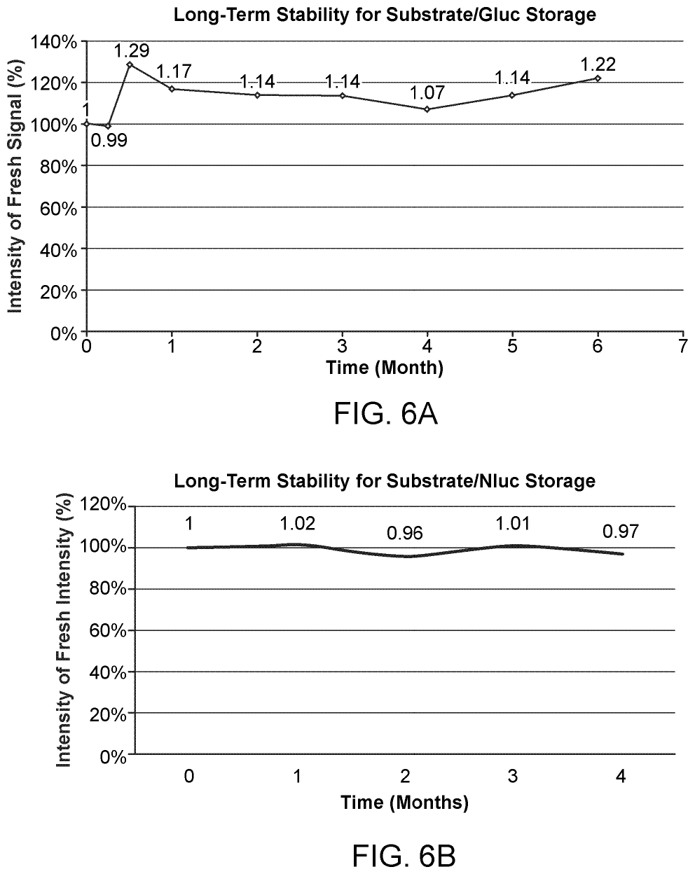

For Gluc/CTZ, 6 months long-term stability at −20° C. showed that no significant signal drop during 6 months shelf life, indicating that the reaction formulation is optimal to protect the CTZ from oxidation. See FIG. 6A.

For Nluc/f-CTZ, 4 months long-term stability at −20° C. showed that no significant signal drop during 4 months shelf life, indicating that the reaction formulation is optimal to protect the f-CTZ from oxidation. More data points are still under shelf life study. See FIG. 6A

### Example 3. NLRT Antibodies

The present invention employs the CooIMPS™ chemistry NLRT antibodies discussed in U.S. Pat. Pub. No. 20180223358), which specifically recognizes and binds to NLRTs at the 3′ end of a growing DNA strand, e.g., after incorporation by a polymerase to the end of a growing DNA chain during sequencing by synthesis (SBS).

### 3.1 NLRT Antibody Modification Sites

In sequencing technology employing un-labeled reserve terminator for incorporation during sequencing, signaling molecules are attached to the NLRT antibodies for the differentiation of different bases. Here in this invention, we selected luciferase as the signal labels on the NLRT antibodies. In order to conjugate the luciferase protein on the NLRT antibodies, multiple labeling sites on the NLRT antibodies can be used: (1) Primary amines (—NH2): these occur on lysine residues and the N-terminus of each polypeptide chain. They are numerous and distributed over the entire antibody. (2) Sulfhydryl groups (—SH): these occur on cysteine residues and exist as disulfide bonds that stabilize the whole-molecule structure. Hinge-region disulfides can be selectively reduced to make free sulfhydryls available for targeted labeling. (3) Carbohydrates (sugars): glycosylation occurs primarily in the Fc region of antibodies (IgG). Component sugars in these polysaccharide moieties that contain cis-diols can be oxidized to create active aldehydes (—CHO) for coupling. (Wild, the Immunoassay Handbook, 4th ed.; Elsevier: Amsterdam, the Netherlands, 2013; Kobayashi and Oyama, Analyst 136:642-651, 2011)

### 3.2 NLRT Antibody-Luciferase Conjugation Methods

A few chemical conjugation approaches for NLRT antibody-luciferase labeling are summarized below:

### 3.2.1. SMCC Chemistry

SMCC linker was used as the cross-linker to link the NLRT antibodies with luciferases. As shown in the scheme in FIGS. 7A and 7B, the —NHS group on the SMCC linker first reacts with the primary amine groups on the Nluc. The NLRT antibodies were treated with reducing agent Tris (2-carboxyethyl) phosphine hydrochloride (TCEP) to open the hinge-region disulfides to generate sulfhydryl groups (—SH). The sulfhydryl groups react with the maleimide group on the other end of the SMCC linker, which links the NLRT antibody with the Nluc.

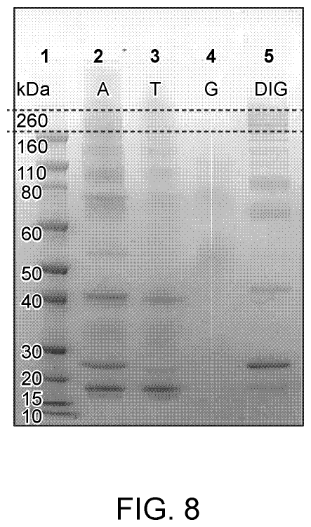

Optimal reaction condition between Nluc and SMCC at Nluc:SMCC=1:8 gave the best Nluc activity and signal intensity. The optimal ratio between Nluc and NLRT antibodies is Nluc:Antibody=1:10. With these conjugation conditions, conjugates between Nluc and NLRT-A, NLRT-T and anti-Digoxin (used as control antibody) were successfully obtained. As shown in FIG. 8, multiple bands between 160-260 KDa were shown in the lanes of NLRT-A, NLRT-T and anti-Digoxin, indicating the presence of Nluc-antibody conjugate product.

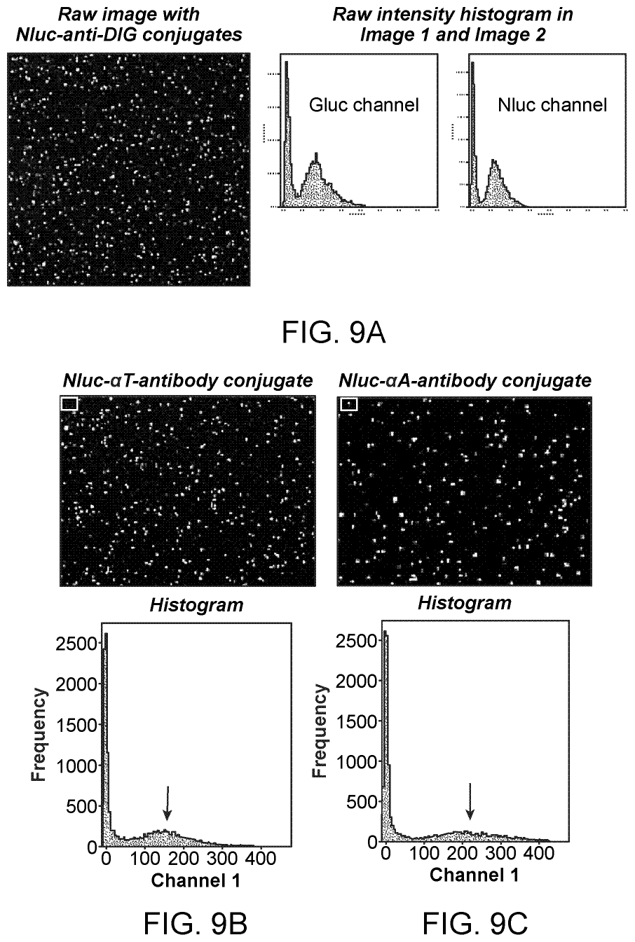

The Nluc_anti-DIG conjugate was also confirmed on the MGI DNBseq E series one-color sequencer imager. The Nluc channel showed similar signal intensity as the control Gluc channel, with a clear peak separation of signal distribution from background, indicating the successful conjugation of Nluc onto the anti-Digoxin antibody. See FIG. 9A

The Nluc_NLRT-A and Nluc_NLRT-T conjugates were also confirmed on the MGI DNBseq E series one-color sequencer imager, as shown in the signal raw imaging and histogram below, indicating the successful conjugation of Nluc onto NLRT-A and NLRT-T antibodies using SMCC linker. NLRT-C and NLRT-G both gave significant lower signal with Nluc conjugated onto the antibodies. See FIGS. 9B and 9C. However, no successful conjugation were obtained between Gluc and NLRT antibodies using the SMCC linker.

### 3.2.2. Traut's Chemistry

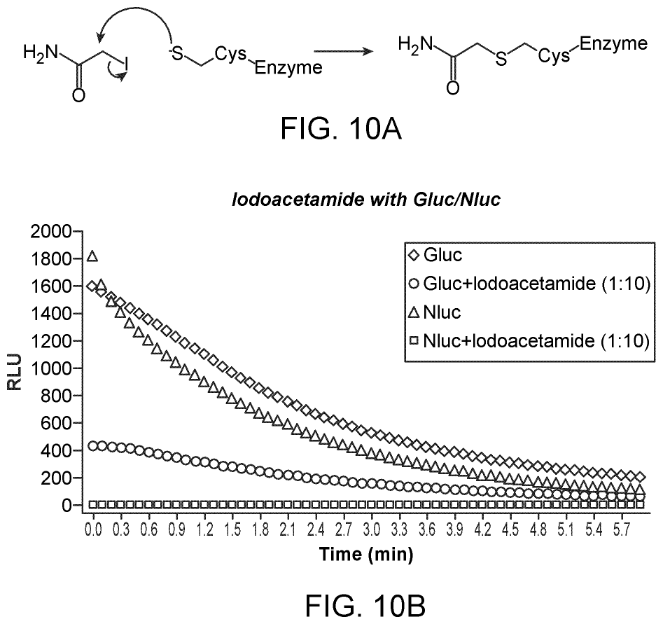

It was noted that the signal of Nluc and Gluc dropped 15%-30% after incubating with SMCC linker. We investigated the detailed luciferase structures to find the reason for the signal drop. According the amino acid sequences of Nluc and Gluc, Nluc has 1 Cysteine, therefore no disulfide bond, and 7 lysines; while Gluc has 10 cysteine, 4 disulfide bonds, 2 free cysteines and 19 lysines. It was suspected that the disulfide bond in Gluc was blocked by the maleimide group on SMCC, which was critical to keep the enzymatic activity for Gluc. To confirm this hypothesis, lodoacetamide was used to incubate with both Gluc and Nluc. Lodoacetamide can bind covalently to the thiol group of cysteine so the protein cannot form disulfide bonds, as illustrated in FIG. 10A. The results showed that Gluc lost 2/3 of signal after reacting with lodoacetamide at Gluc:lodoacetamide at 1:10 ratio at RT for 30 min; while Nluc lost all signal after reacting with lodoacetamide at Nluc:lodoacetamide at 1:10 ratio at RT for 30 min. The results (shown in FIG. 10B) indicated that there were free —SH group on both Gluc and Nluc, and blocking these —SH group (by lodoacetamide) leads to loss of enzyme activity. And SMCC linker has maleimide group which can react with the free —SH group on the Gluc/Nluc. This was the reason why there is signal loss after incubating the SMCC with Nluc/Gluc which leads to low signal intensity for the final antibody conjugates.

Other conjugation methods were also attempted to avoid the side reactions from SMCC. Traut's reagent (Thermo Fisher Inc.) appeared to be a better linker for Gluc/Nluc and have minimal impact on —SH groups on the luciferases. The conjugation chemistry scheme is shown in FIG. 11A. Traut's reagent reacts with the primary amine group on Gluc/Nluc to generate —SH group on luciferase; while the NLRT antibodies are treated with SMCC linkers to react through the primary amine on antibody and the —NHS group on the SMCC (FIG. 11B). After purification of each reaction, the luciferase after Traut's treatment and the antibody after SMCC treatment were mixed together to react through the —SH group on the luciferase and maleimide on the SMCC group on antibody.

Both Gluc and Nluc show improved signal after Traut's modifications. As shown in FIGS. 11C and 11D, 1:1 Gluc/Nluc to Traut's molar ratio gives the highest signal. After Traut's modification, Gluc signal has been improved 60% and Nluc signal has been doubled. Beside signal evaluation, stability study had also been investigated. The Gluc/Nluc_Traut's conjugates have been incubated at 50° C. or 70° C. for 1 hour for thermostability test. For Gluc, the Traut's modification leads to worse stability at 50° C. or 70° C. For Nluc, Traut's modification (1:1 ratio) helps to improve stability and signal for about 30% at 50° C. At 70° C., both modified or unmodified Nluc cannot survive 70° C.

Nluc-anti-Digoxin and Nluc-NLRT antibodies conjugate had been successfully obtained through the Traut's method. Different Nluc to Traut's ratio have been tested at 1:15, 1:2 and 1:1. With higher ratio of Traut's reagent, the conjugate gave higher signal. However, the conjugate signal was similar to the SMCC method, with no further signal enhancement as discussed in the hypothesis and shown in the initial data above. Gluc-NLRT antibody conjugates were also obtained, but signal intensity was significantly lower than the Nluc-NLRT antibody conjugates.

### 3.2.3. Reducing Chemistry

In order to obtain more copies of luciferase on each antibody for higher luciferase signal per conjugate, different reducing agents (TCEP, Mercaptoethylamine-HCl) and other linkers (SMCC, Traut's) at different ratios with the NLRT antibodies were investigated.

As shown in the data below, high conc. TCEP (1:1500), lower ratio of TCEP (1:150), Mercaptoethylamine-HCl, and other cross-linkers such as SMCC, Traut's have been tested. The 1500:1 ratio of TCEP gave the highest —SH labeling degree and highest signal from the sequencer imager among all reducing methods. See FIG. 12A-12C.

### 3.3. Secondary Antibody Labeling Method

Beside direct labeling NLRT antibodies with luciferase, secondary antibody labeling methods have also been attempted. Biotin-labeled secondary antibodies (i.e., anti-primary goat IgG antibodies) specific for the isotype NLRT-antibodies were used to target and label the NLRT-antibodies specifically. The Streptavidin-labeled Gluc was used for signal labeling. Due to the secondary layer labeling scheme, more Gluc can be bound to each NLRT-antibody, thus leading to higher signal intensity. As shown in FIG. 15, image 1 was a control image with Biotin-labeled dCTP incorporated onto the DNA strand, then labeled with Streptavidin-labeled Gluc. Image 2-4 are unlabeled dNTP incorporated onto the DNA strands, followed by NLRT antibodies binding, biotin-labeled secondary antibody binding, then Streptavidin-labeled Gluc binding. About double of signal intensity was obtained compared to the control scheme using the secondary antibody labeling scheme. However, the drawback of this scheme is more reaction steps in the each sequencing cycle which leads to longer sequencing time.

### 3.4. NLRT Antibody-Luciferase Fusion Protein

In addition, fusions directly linking recombinant antibody fragments, e.g., single-chain Fv fragments (scFvs) with reporter proteins (Skerra and Plückthun, Science 240:1038-1041, 1988; Bird et al., Science 242:423-426, 1988; Huston et al., Methods Enzymol 203:46-88, 1991; Ahmad et al., Clin. Dev. Immunol. 2012:1, 2012) may be used. For example, photoproteins with bioluminescent properties, e.g., luciferases and aequorin, may be used as reporter proteins in fusion proteins with antibody fragments, epitope peptides and streptavidin, for example (Oyama et al., Anal Chem 87:12387-12395, 2015; Wang et al., Anal Chim Acta 435:255-263, 2001; Desai et al., Anal Biochem 294:132-140, 2001; Inouye et al., Biosci Biotechnol Biochem 75:568-571, 2011).

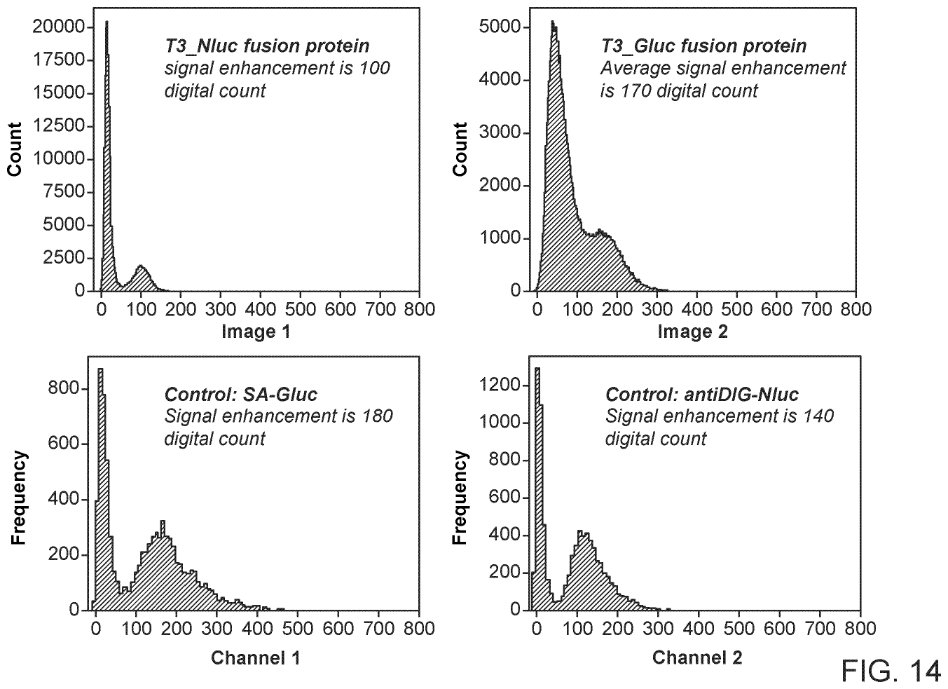

Some initial attempt of using NLRT Antibody-Luciferase fusion protein has been done. As shown in the FIG. 14, the NLRT-T-Gluc and NLRT-T-Nluc fusion proteins show good binding capacity to the unlabeled RT. More work will be done to improve the performance of the fusion proteins.

### 3.5. NLRT-Antibody-Biotin Labeling

Biotin conjugates with NLRT-antibodies (A, T, C, G and 2nd Antibody) have been developed using the EZ-NHS-S-S-Biotin linker (Thermo Fisher). The EZ-NHS-S-S-Biotin linker reacted with the NLRT-antibodies through their primary amine groups. Then Streptavidin-labeled Gluc was used to label the NLRT-antibodies. The optimal signal intensity was obtained at the ratio of antibody:biotin linker=1:10 ratio. See FIG. 15.

### Example 4. Bioluminescence-Based Sequencing Scheme

NLRT-antibody-Luciferase conjugates may be used in nucleic acid sequencing methods and find particular use in one-color (also called one-channel) sequencing.

According to one such method, an array is provided that comprises single-stranded nucleic acid templates disposed at positions on a surface. Sequencing by extension, or SBS, is performed in order to determine the identity of nucleotides at detection positions in nucleic acid templates in multiple sequencing cycles by: (i) binding (or incorporating) an unlabeled complementary nucleotide (NLRT) to a nucleotide at a detection position, (ii) labeling the NLRT by binding to it a directly or indirectly labeled affinity reagent that specifically binds to such an NLRT; (iii) detecting the presence or absence of a signal(s) associated with the complementary NLRT at the detection position, the signal resulting from the label (e.g., a luciferase signal); wherein (1) detecting a first signal and not a second signal at the detection position identifies the complementary NLRT as selected from NLRT-A, NLRT-T, NLRT-G and NLRT-C;
(2) detecting the second signal and not the first signal at the detection position identifies the complementary NLRT as an NLRT selected from NLRT-A, NLRT-T, NLRT-G or NLRT-C that is different from the NLRT selected in (1);
(3) detecting both the first signal and the second signal at the detection position at different times identifies the complementary NLRT as an NLRT selected from NLRT-A, NLRT-T, NLRT-G and NLRT-C that is different from nucleotides selected in (1) and (2); and
(4) detecting neither the first signal nor the second signal at the position identifies the complementary NLRT as an NLRT selected from NLRT-A, NLRT-T, NLRT-G and NLRT-C that is different from the nucleotides selected in (1), (2) and (3); and (iii) deducing the identity of the nucleotide at the detection position in the nucleic acid template based on the identity of the complementary NLRT.

An example of bioluminescence-based sequencing scheme making use of the NLRT-antibody-Luciferase conjugates is shown in FIG. 16.

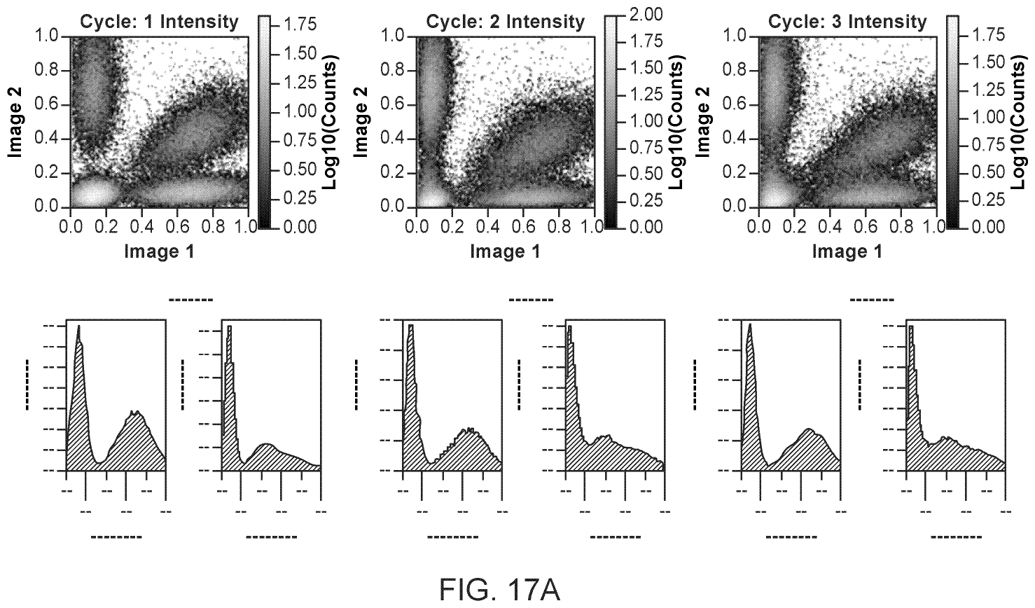

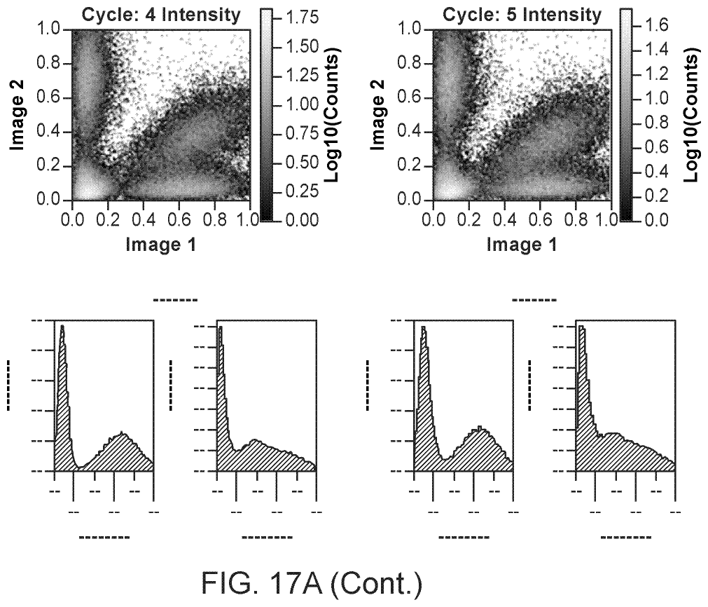

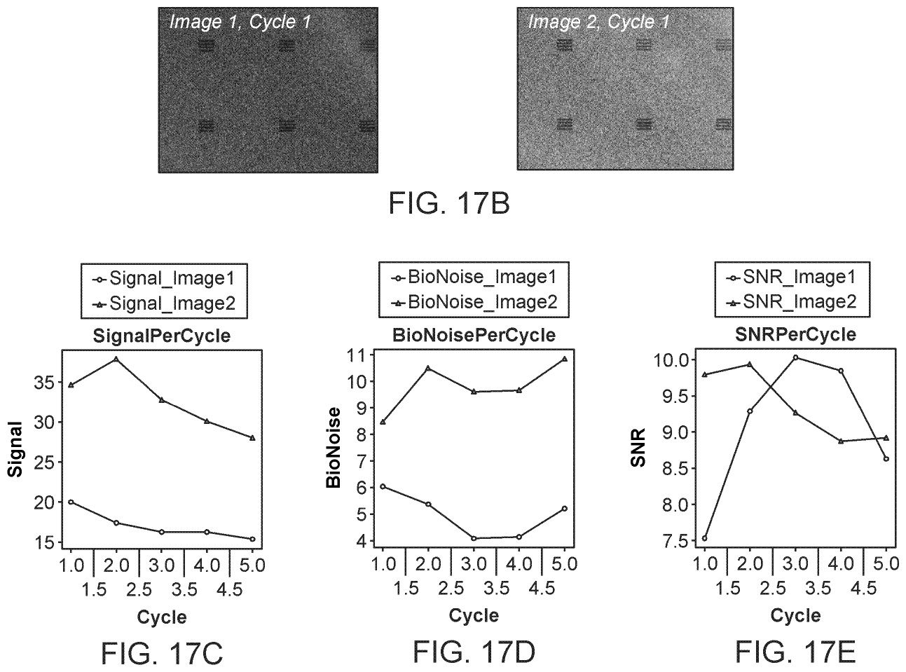

Five cycle sequencing data was obtained. Good signal separation from background was obtained, as shown in signal histogram (FIG. 17A, the bottom panels). A good base separation was also shown in the scattering plot (FIG. 17A, the top panels), indicating good signal specificity from each NLRT-antibody-luciferase conjugates. The signal, noise, and SNR plot for each cycle was also shown in FIG. 17C-17E. Image 1 (signal generated by Nluc) image 2 (signal generated by Gluc) of cycle 1 are shown in FIG. 17B.

### Example 5. Bioluminescence-Based Sequencing Scheme with Selective Deactivation

In each cycle a mixture of NLRTs were incubated with the DNA template to be sequenced in the presence of a DNA polymerase. These NLRTs include 7-deaza-7-biotin-linker-3′-azidomethyl-dATP, 5-biotin-linker-3′-azidomethyl-dTTP, 5-digoxin-linker-3′-azidomethyl-dTTP, 5-digoxin-linker-3′-azidomethyl-dCTP, and 3′-azidomethyl-dGTP. Streptavidin-conjugated Gluc (recognizing biotin-labeled nucleotides A and T) was added to the sequencing flow cell followed by adding substrate CTZ. Bioluminesnce signals from the cleavage of CTZ by the Gluc was collected. 100 mM DTT was then flown into the sequencing flow cell, which deactivated Gluc. Subsequently, Nluc-conjugated anti-Digoxin antibody was added to the flow cell to bind the Digoxin-labeled nucleotides (T and C). No signals for G were detected as expected.

At the end of the first cycle, the azidomethyl group was cleaved off the nucleotide at the end of the GDS. The biotin or the digoxin labels were released and washed off the flow cell. The next cycle began with adding a new mixture of the NLRTs.

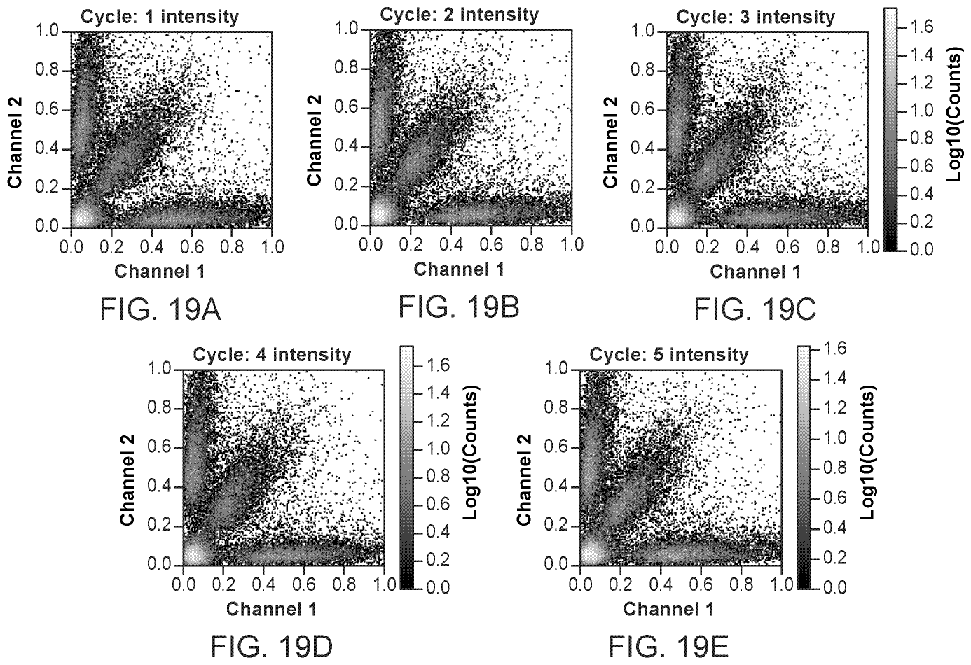

FIG. 19A-19E show the signals from the first five sequencing cycles. The results show good signal separation from background. In each signal scattering plot, the X axis indicates the normalized signal intensity from Image 1, and the Y axis indicates the normalized signal intensity from Image 2. There were four (4) well separated signal groups obtained from this scattering plots. The one on the X axis (median=0.5, 0) is from the biotin-labeled nucleotide A. The one on the Y axis (0, median=0.5) is from the Digoxin-labeled nucleotide C. Signal from the (0, 0) position indicates signal from nucleotide G, which was unlabeled. Signal from the (0.5, 0.5) position in the 45 degree is signal from the nucleotide T since it was half labeled with Biotin and half with Digoxin. So one half of the Ts was lighted up in image 1 and the other half lighted up in Image 2. In each cycle of the signal scattering plot, these four (4) signal groups were well differentiated from each other, rendering it feasible for correct base call for that cycle.

### INCORPORATION BY REFERENCE

Each and every publication and patent document referred to in this disclosure is incorporated herein by reference in its entirety for all purposes to the same extent as if each such publication or document was specifically and individually indicated to be incorporated herein by reference.

While the invention has been described with reference to the specific examples and illustrations, changes can be made and equivalents can be substituted to adapt to a particular context or intended use as a matter of routine development and optimization and within the purview of one of ordinary skill in the art, thereby achieving benefits of the invention without departing from the scope of what is claimed and their equivalents.

1. A method of corresponding positions on an array with differently labeled affinity reagents immobilized at the positions comprising
i) providing an array of positions wherein the array comprises first positions and second positions,
wherein a first affinity reagent is immobilized at least some of the first positions and a second affinity reagent is immobilized at least some of the second positions;
wherein the first affinity reagent is associated with a first luciferase polypeptide and the second affinity reagent is associated with a second luciferase polypeptide,
wherein the first luciferase polypeptide can react with a first substrate to generate a first luminescent signal,
wherein the second luciferase polypeptide can react with a second substrate to generate a second luminescent signal,
wherein the first substrate is orthogonal to the first luciferase polypeptide and the second substrate is orthogonal to the second luciferase polypeptide, and
wherein the first luciferase polypeptide does not cross react with the second substrate and the second luciferase polypeptide does not cross react with the first substrate,
ii) contacting the array with the first substrate and detecting the first luminescent signal at positions at which the first affinity reagent is immobilized
iii) contacting the array with the second substrate and detecting the second luminescent signal at positions at which the second affinity reagent is immobilized, and
iii) determining a position is immobilized with the first affinity reagent if the first luminescent signal is detected at said position, or
determining at a position is immobilized with the second affinity reagent is immobilized if the second luminescent signal is detected at said position, thereby corresponding the positions on the array with differently labeled affinity reagents.

2. The method of claim 1, wherein the first substrate is f-CTZ and the first luciferase polypeptide is Nluc.

3. The method of claim 1, wherein the second substrate is f-CTZ and the second luciferase polypeptide is Nluc.

4. The method of claim 1, wherein the first substrate is coeleterazine (CTZ), and the second substrate is f-CTZ.

5. The method of claim 4, wherein the first luciferase polypeptide is a Gaussia luciferase polypeptide (Gluc) and the second luciferase polypeptide is a NanoZac luciferase polypeptide (Nluc).

6. The method of claim 1, wherein the array comprises third positions and fourth positions, a third affinity reagent is immobilized at some of the third positions and a fourth affinity reagent is immobilized at least some of the fourth positions,
wherein the third affinity reagent is associated with both the first luciferase polypeptide and the second luciferase polypeptide, and
wherein the fourth affinity reagent is associated with neither luciferase polypeptide,
wherein determining the first affinity reagent is immobilized at a position if the first luminescent signal is detected from the position,
determining the second affinity reagent is immobilized at a position if the second luminescent signal is detected from the position,
determining the third affinity reagent is immobilized at a position if both the first luminescent signal and the second luminescent signal are detected from the position,
determining the fourth affinity reagent is immobilized at a position if neither the first nor the second luminescent signal is detected from the position.

7. The method of claim 1, wherein the first luminescent signal is detected before the second luminescent signal is detected, and
wherein a deactivation agent is added after detection of the first luminescence but before the detection of the second luminescent signal, and
wherein the deactivation agent selectively deactivates the first luciferase polypeptide.

8. The method of claim 7, wherein the deactivation agent is dithiothreitol (DTT).

9. The method of claim 1, wherein the first or the second affinity reagent specifically binds to a 3′-O-reversible terminator deoxyribonucleotide comprising a nucleobase selected from the group consisting of adenine (A), cytosine (C), guanine (G), thymine (T), and analogs thereof.

10. The method of claim 9, the method further comprises identifying a type of 3′-O-reversible terminator deoxyribonucleotide associated with if the affinity reagent for the type of 3′-O-reversible terminator deoxyribonucleotide is determined to be immobilized at said position.

11. The method of claim 1, wherein the first luciferase polypeptide is conjugated to the first protein via a linker, and/or
wherein the second luciferase polypeptide is conjugated to the second protein via a linker.

12. The method of claim 11, wherein the linker is an SMCC linker.

13. The method of claim 1, wherein the first luciferase polypeptide comprises a —SH group linked to a maleimide group on a SMCC linker, and a —NETS group of the SMCC linker is linked to a —NH2 group on the first protein; and/or
wherein the second luciferase polypeptide comprises a —SH group linked to the maleimide group on the SMCC linker, and a —NETS group of the SMCC linker is linked to a —NH2 group on the second protein.

14. The method of claim 13, wherein the SH group on the first luciferase is generated by treating the first luciferase polypeptide with a cyclic thioimidate compound.

15. The method of claim 13, wherein the SH group on the second luciferase is generated by treating the second luciferase polypeptide with a cyclic thioimidate compound.

16. The method of claim 14, wherein the cyclic thioimidate compound is 2-iminothiolane.

17. A kit for performing sequencing, the kit comprising:
(1) a first protein that is associated with a first luciferase polypeptide,
wherein the first luciferase polypeptide can specifically cleave a first substrate to generate a first luminescent signal,
(2) a second protein that is associated with a second luciferase polypeptide,
wherein the second luciferase polypeptide can specifically cleave a second substrate to generate a second luminescent signal,
(3) a first substrate, and
(4) a second substrate;
wherein the first luciferase polypeptide does not cross react with the second substrate and the second luciferase polypeptide does not cross react with the first substrate.

18. The kit of claim 17, further comprising a third protein and a fourth protein, wherein the third protein is associated with both the first luciferase polypeptide and second luciferase polypeptide, and wherein the fourth protein is associated with neither the first nor the second luciferase polypeptide.

19. The kit of claim 17, wherein the first luciferase polypeptide is Gluc and the second luciferase polypeptide is Nluc.

20. The kit of claim 17, wherein the first substrate is coeleterazine (CTZ).

21. The kit of claim 17, wherein the second substrate is f-CTZ, having a structure shown below:

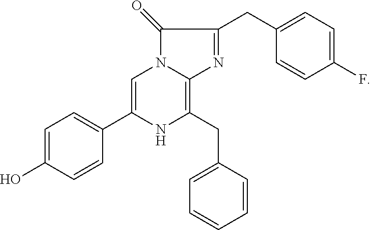 [SMILES: O=c1c(Cc2ccc(F)cc2)nc2c(Cc3ccccc3)[nH]c(-c3ccc(O)cc3)cn1-2]

22. The kit of claim 17, wherein the kit further comprises a plurality of a 3′-O-reversible terminator deoxyribonucleotides, wherein each 3′-O-reversible terminator deoxyribonucleotide comprises a different nucleotide base.

23. The kit of claim 17, wherein the first substrate or the second substrate is present in a stock buffer, wherein the stock buffer is selected from the group consisting of ethanol, propylene glycol, methanol, DMSO, and any combinations thereof.

24. The kit of claim 23, wherein the stock buffer is a mixture of 50% v/v ethanol with 50% v/v propylene glycol.

25. The kit of claim 17, wherein the kit further comprises an antioxidant, wherein the antioxidant can prevent oxidation of the first substrate, the second substrate, or both the first and the second substrates.

26. A method of producing a luciferase-antibody conjugate, comprising:
(1) providing (i) an antibody that specifically recognizes a 3′-O-reversible terminator deoxyribonucleotides comprising a nucleobase that is selected from the group consisting of adenine (A), cytosine (C), guanine (G), thymine (T), and analogs thereof; and (ii) a luciferase polypeptide,
(2) contacting the luciferase polypeptide with 2-iminothiolane under conditions that generate an —SH group on the luciferase polypeptide, thereby producing a luciferase polypeptide comprising an —SH group.
(3) contacting the antibody with an SMCC, wherein the —NETS group of the SMCC is linked to an —NH2 group on the antibody, thereby producing an SMCC-linked antibody having a maleimide group, and
(4) contacting the luciferase polypeptide comprising the —SH group with the SMCC-linked antibody under conditions suitable for protein conjugation thereby forming the luciferase-antibody conjugate.

27. The method of claim 26, wherein the luciferase polypeptide is a Gluc or a Nluc.

28. The method of claim 26, wherein the luciferase polypeptide is conjugated to the antibody on a primary amine, a sulfhydryl group, or a carbohydrate.

29. The method of claim 26, wherein the conjugation occurs in a reaction buffer, wherein the reaction buffer comprises Tris-HCL, NaCl, Tween 20, sodium ascorbate, and PEG 3350.

30. The method of claim 26, wherein the molar ratio between the luciferase polypeptide and the antibody ranges from 1:3 to 1:20.

31. The method of claim 26, wherein the antibody is treated with a reducing agent before conjugation with the luciferase polypeptide,
wherein the molar ratio of the antibody to the reducing agent ranges between 1:1 and 1:5.

32. The method of claim 31, wherein the reducing agent is TCEP or Mercaptoethylamine-HCl.


SEQUENCE LISTING (2 sequences)

SEQ ID NO: 1 - protein, 175 aa, Artificial Sequence
  Description of Artificial Sequence Synthetic polypeptide
MKPTENNEDFNIVAVASNFATTDLDADRGKLPGKKLPLEVLKELEANARKAGCTRGCLICLSHIKCTPKMKKFIPGRCHTYEGDKESAQGGIGEAIVDIPEIPGFKDLEPLEQFIAQVDLCVDCTTGCLKGLANVQCSDLLKKWLPQRCATFASKIQGQVDKIKGAGGDHHHHHH

SEQ ID NO: 2 - protein, 171 aa, Artificial Sequence
  Description of Artificial Sequence Synthetic polypeptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA